(19) United States
(12) Patent Application Publication
(10) Pub. No.: US 2024/0369537 A1
(43) Pub. Date: Nov. 7, 2024

(54) In Vitro Cell Co-Cultures of Neuronal Cells, Astrocytes, and Oligodendrocytes

(71) Applicant: KATHOLIEKE UNIVERSITEIT LEUVEN, Leuven (BE)

(72) Inventors: Karan Ahuja, Leuven (BE); Rodrigo Furtado Madeiro da Costa, Leuven (BE); Cathérine Verfaillie, Leuven (BE)

(21) Appl. No.: 18/692,616

(22) PCT Filed: Sep. 20, 2022
(86) PCT No.: PCT/EP2022/076103

§ 371 Date: Mar. 15, 2024

(30) Foreign Application Priority Data
Sep. 20, 2021 (EP) 21197737.6

(51) Int. Cl. G01N 33/50; C12N 5/079; C12N 5/0793

(52) U.S. Cl. CPC G01N 33/5058; C12N 5/0619; C12N 5/0622; C12N 2501/105; C12N 2501/11; C12N 2501/115; C12N 2501/12; C12N 2501/13; C12N 2501/135; C12N 2501/155; C12N 2501/999; C12N 2506/45

(57) ABSTRACT

The invention relates to an in vitro cell culture comprising neuronal cells derived from iPSC, optionally with an NGN2 transgene, astrocytes derived from iPSC with a SOX9 transgene, and oligodendrocytes derived from iPSC with a SOX10 transgene.

### FIELD OF THE INVENTION

The invention relates to co-cultures of brain cells.

### BACKGROUND OF THE INVENTION

Understanding the neurological disease progression and investigating role of novel drugs using available animal-based model system and post-mortem brain tissue has remained a major challenge for neuroscientists. The combination of stem cell biology with genome engineering has revolutionized the field of disease modelling by providing tools to scientists to understand neurodevelopment and disease pathogenesis in less or more complex environments.

### SUMMARY OF THE INVENTION

The present invention discloses a two-dimensional or three-dimensional triculture system consisting of human induced pluripotent stem cell derived neurons and glial cells to study the differentiation and co-maturation of cells in complex environment. Growth-factor based and transcription factor overexpression-based protocols were used to differentiate neurons (cortical and inducible neurons) and glial cells (astrocytes and oligodendrocytes), characterise their monocultures and optimize parameters like media composition, cell type ratio, cell densities and cell seeding strategy. The established human triculture system display complex cell arrangement with different cell morphologies of neurons, astrocytes, and oligodendrocytes, compared to their monoculture counterparts. The present invention further demonstrates efficient neuron maturation in terms of expression of deep layer neuron markers as well as neuronal activity (e.g., neuronal spikes and bursts) in triculture system, compared to neuron monoculture system. The present invention provides an all-human iPSC-derived neural model for disease modelling, drug development in neuroscience and reduces the need of animal-based models in biomedical research.

Glial cells such as astrocytes and oligodendrocytes play besides their well-known feature of providing protection and support to neurons, also a vital role not only in brain development, but also brain homeostasis and disease progression in several neurological disorders. Thus, in vitro models that recapitulate neuron-glia interactions allow to understand the crosstalk between these cells and gain more insights in their role in neurological disorders. The coalescence of genome engineering and iPSC technology can be used to develop better in vitro models. Although differentiation protocols, based on growth factor mediated fate specification produced mature neurons and glial cells, they are time-consuming and result in low purity of the progeny cells [Shi et al. Nat. Protoc. 7, 1836-1846 (2012)]. Recent improvements in transcription factor-based cell differentiation, can accelerate the production of neurons and glial cells. For instance, SOX9, SOX10, and NGN2 transcription factor overexpression was shown to generate functional astrocytes, oligodendrocytes, and neurons respectively [Neyrinck et al. Stem Cell Rev. Reports 1-19 (2021); Garcia-León et al. (2020) cited above].

In the present invention a combination of growth factor-based and transcription factor-based differentiation methods are used to generate neurons, astrocytes, and oligodendrocytes; coculture conditions are optimised to create a human neuron, astrocyte, and oligodendrocyte triculture system.

The present invention generates and characterises neurons, astrocytes, and oligodendrocytes derived from iPSCs using overexpression of specific transcription factors and/or small molecules-based differentiation protocols.

iPSC lines with and without an inducible transcription factor are used to generate neurons (cortical and inducible), astrocytes, and oligodendrocytes. Newly differentiated cells were characterized by RT-qPCR, flow cytometry and immunofluorescence microscopy.

The present invention defines co-culture media that maintain the viability of iPSC-derived neurons, astrocytes and oligodendrocytes in monocultures and co-cultures.

The differentiated cells are allowed to grow separately in two different mediums for one week. Cell viability of the differentiated progeny is evaluated by RT-qPCR and immunofluorescence microscopy to determine the efficiency of both media.

The present invention develops a triculture, by testing seeding density and strategy, cell type ratio and identifies antibodies that can be used to follow the cells in tricultures using the selected co-culture medium composition.

The selected medium are to establish individual cell cultures and tricultures containing neurons, astrocytes, and oligodendrocytes.

The present invention characterizes cell-cell interactions and co-differentiation of cells towards the respective mature phenotype.

Maturation of cells in the human triculture system is evaluated using immunofluorescence microscopy and a multielectrode array system.

### DETAILED DESCRIPTION

Figure Legends

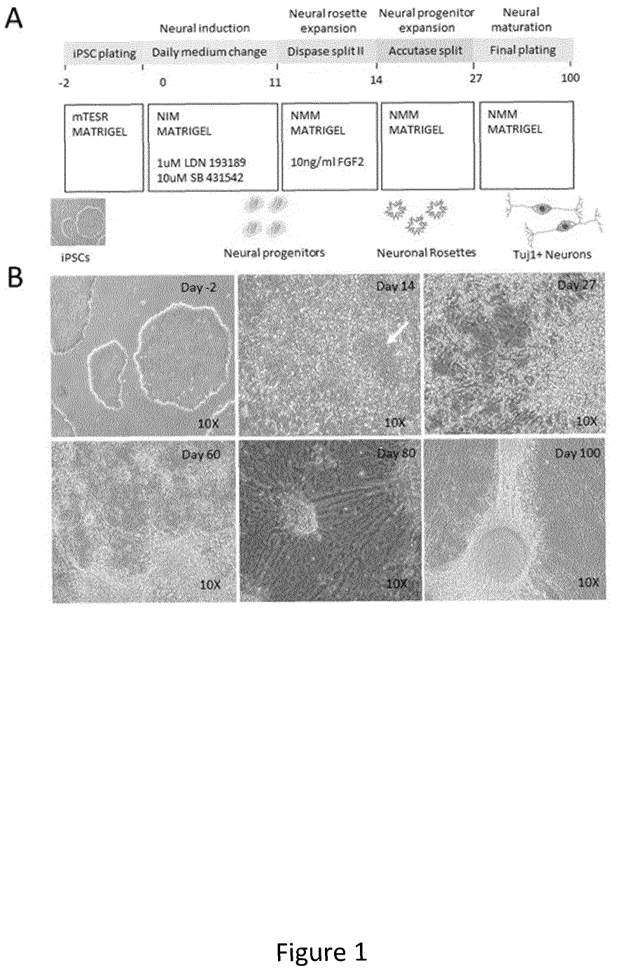

FIG. 1: Overview of cortical neuron differentiation and maturation. A.

Timeline of neuronal differentiation protocol. Neural induction medium (NIM), Neural Maintenance Medium (NMM), Fibroblast growth factor 2 (FGF). B, Brightfield microscopy pictures taken from cell cultures at different time points. From left to right: Day −2=SIGi001-A iPSC colony, Day 14=Neural rosettes (indicated by white arrow) in neural expansion phase, Day 27=Neural progenitor cells. In the lower panels, light microscopy images of iPSC progeny in 2D culture on Day 60, 80 and 100.

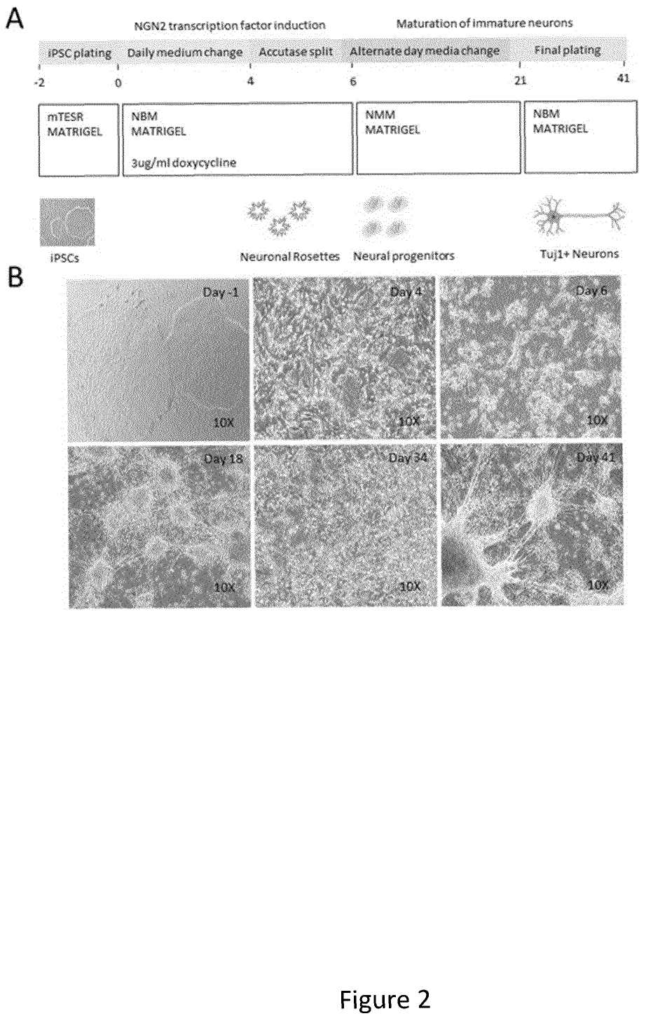

FIG. 2: Overview of induced neuron differentiation and maturation.

A Timeline of induced neuron differentiation protocol for the BIONi010-C+NGN2-I7-26 iPSC line (iNGN2 cell line). Neurobasal medium (NBM), Neural Maintenance Medium (NMM). B, Brightfield microscopy pictures taken from cultures at different time points. From left to right: Day −2=BIONi010-C+NGN2-I7-26 iPSC, Day 4, and Day 6=Doxycycline induced iNGN2 expression, Day 18=Neural progenitor cells; Day 34 and 41=neuronal maturation.

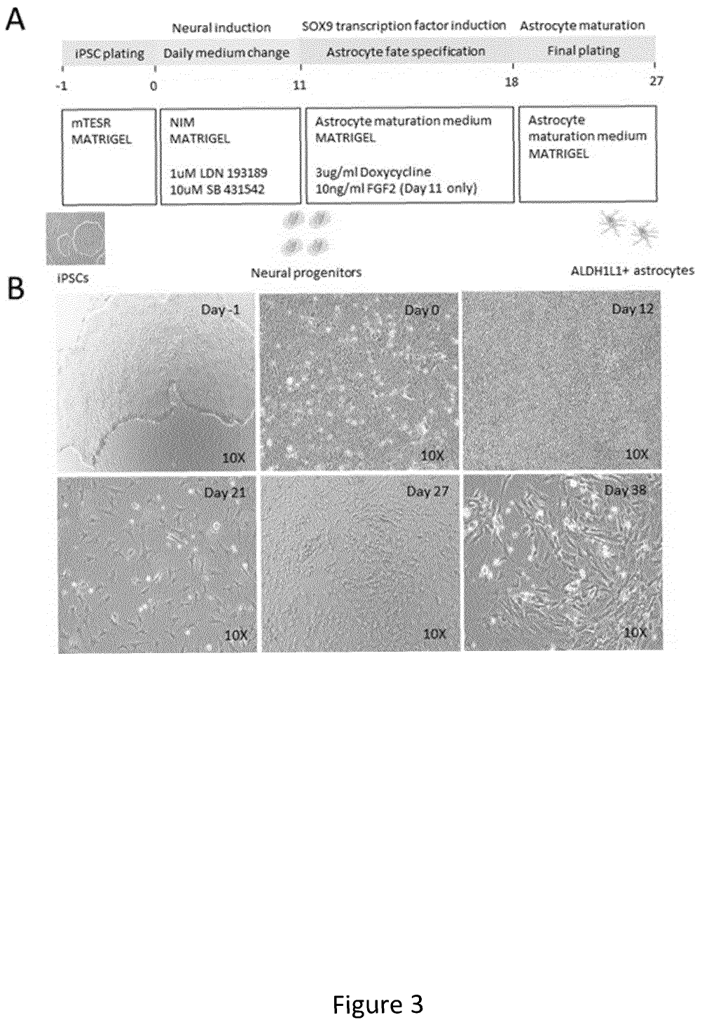

FIG. 3 Overview of iSOX9-iPSC astrocyte differentiation and maturation.

A. Timeline of astrocyte differentiation protocol. Neural induction medium (NIM). B, Brightfield microscopy pictures taken from cultures at different time points. From left to right: Day −1=iSOX9 iPSCs, Day 0-12=Neural induction phase, Day 21=Immature astrocytes; Day 27 and 38=Maturation of astrocytes in culture.

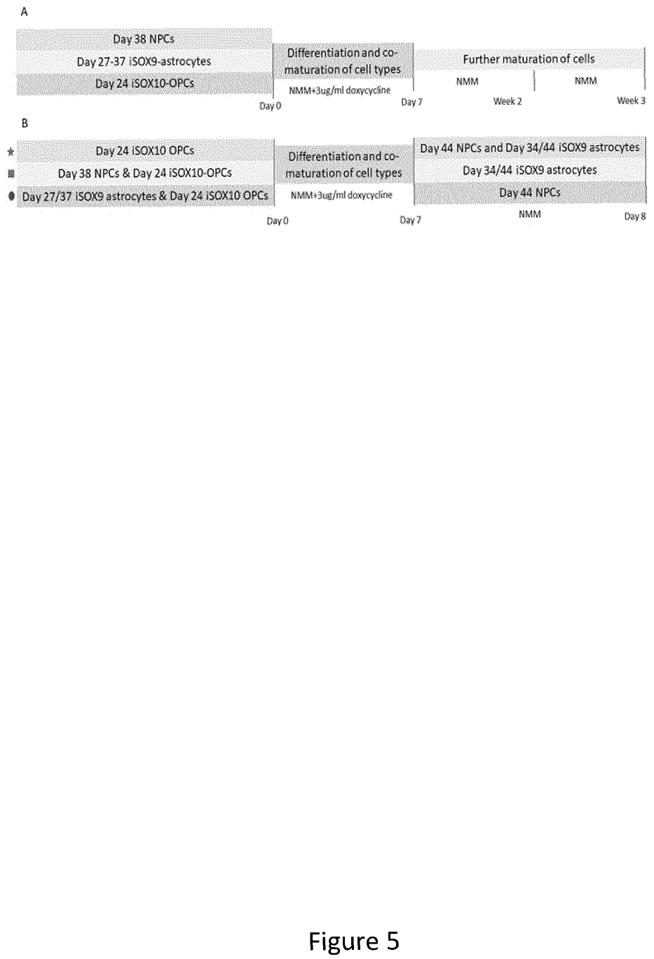

FIG. 5 Overview of oligodendrocyte differentiation and maturation from iSOX10 iPSCs.

A. Timeline of oligodendrocyte differentiation protocol. B, Brightfield microscopy pictures taken from cultures at different time points. From left to right: Day −2=iSOX10 iPSCs, Day 0=Start of neural induction phase, Day 21=OPCs (indicated by white arrow).

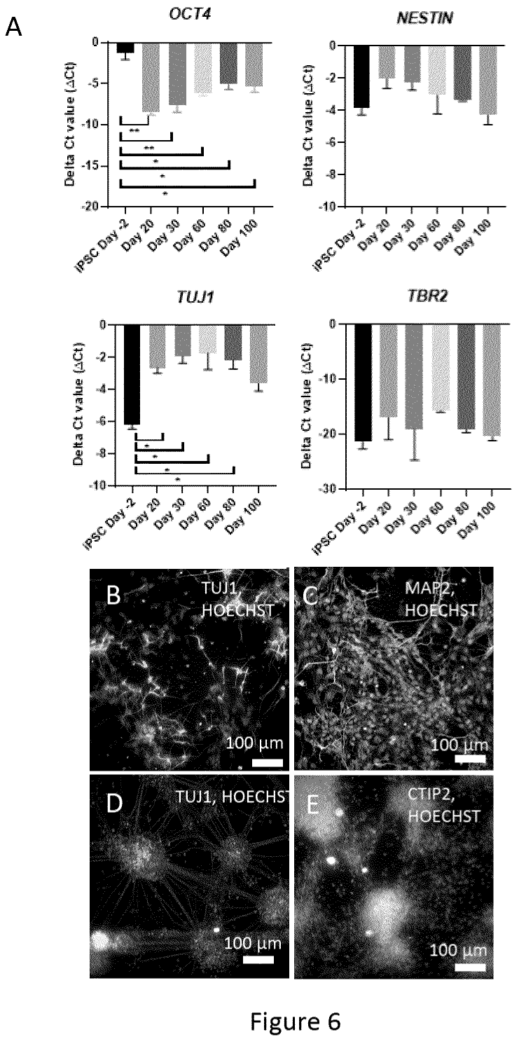

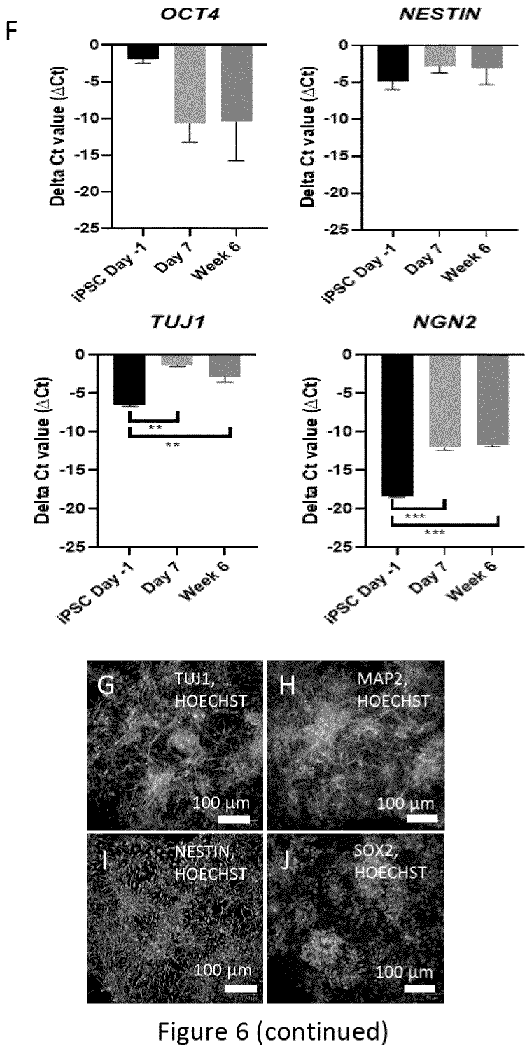

FIG. 6: Characterization of neuronal progeny from SIGi001-A iPSC and BIONi010-C iNGN2 iPSC

SIGi001-A iPSCs were differentiated to cortical neurons (panels A-E) using double SMAD inhibition and maintained in neural maintenance medium until day 100. BIONi010-C iNGN2 iPSCs (panels F-J) were differentiated by NGN2 transcription factor overexpression for which doxycycline was added from day 0-day 6. Subsequently cells were maintained in neural maintenance medium and neurobasal medium until week 6. Cells were analyzed by qRT-PCR on the days indicated, and fluorescence immunology microscopy on day 40 and 60 for cortical neuron precursor cells and day 21 for iNGN2 neural progenitors was done. (A): qRT-PCR analysis of iPSC on day −2 and neuronal differentiated progeny on days 20, 30, 60, 80 and 100 after starting differentiation, and transcript levels compared with the housekeeping gene GAPDH (data shown as delta CT). N=2 biological replicates with 3 technical replicates. Data are shown as mean±SD; One-Way ANOVA, multiple comparisons with Bonferroni correction *: p<0.05; **: p<0.01; ***: p<0.001. (B-C) Day 40 SIGi001-A NPCs were stained with antibodies against TUJ1 and MAP2; (D-E) Day 60 neural progenitors with TUJ1 and CTIP2, as well as Hoechst to identify the nuclei. Scale bars: 100 μM. Representative image for 33 images each for 3 wells, taken per differentiation and 2 biological replicates. (F) RT-qPCR analysis of BIONi010-C iNGN2 iPSC on day −1 and neuronal differentiated progeny on day 7, and 6 weeks after starting differentiation, and transcript levels compared with the housekeeping gene GAPDH (data shown as delta CT). N=2 biological replicates and 3 technical replicates. Data are shown as mean±SD; One-Way ANOVA, multiple comparisons with Bonferroni correction: p<0.05; **: p<0.01; ***: p<0.001. (G-J) Day 21 BIONi010-C iNGN2 neuronal progeny were stained with antibodies against TUJ1, MAP2, NESTIN and SOX2, as well as Hoechst to identify the nuclei. Scale bars: 100 μM. Representative image for 33 images each for 3 wells, taken per differentiation and 2 biological replicates.

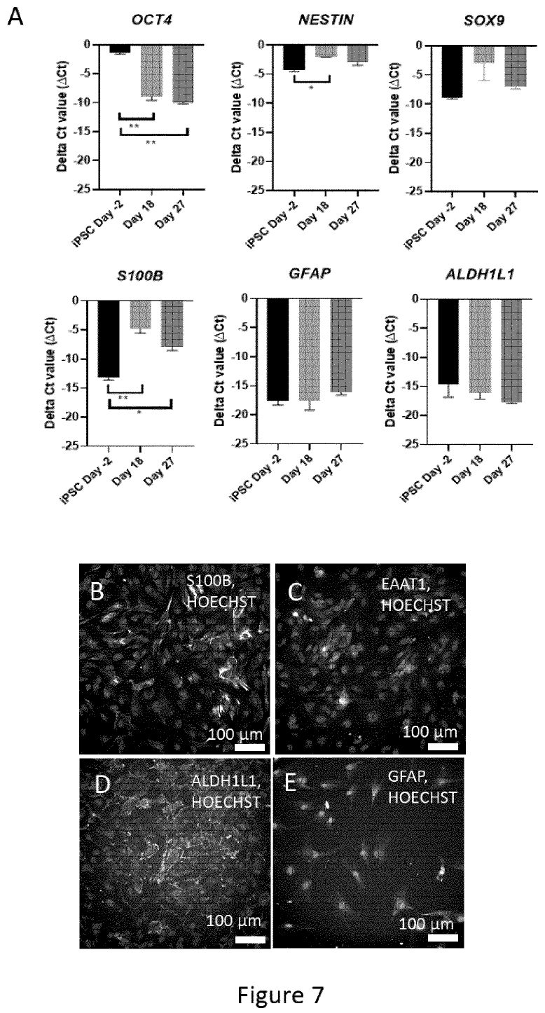

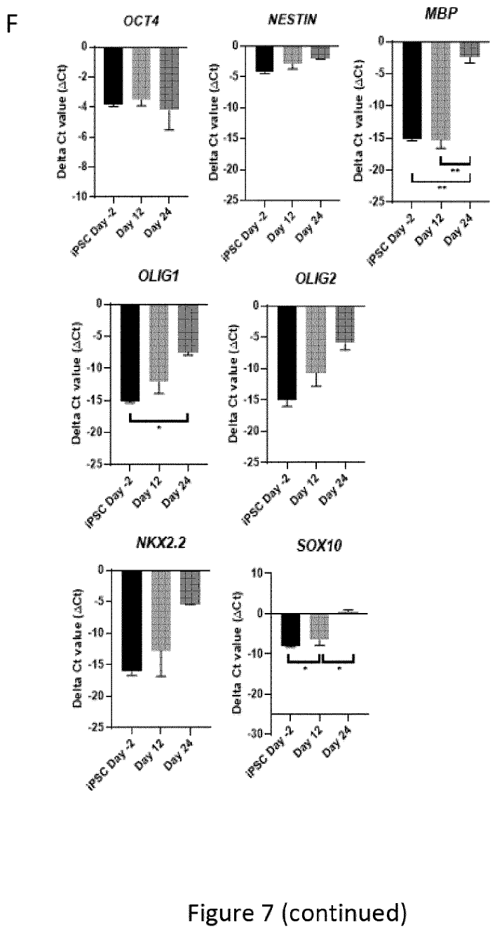

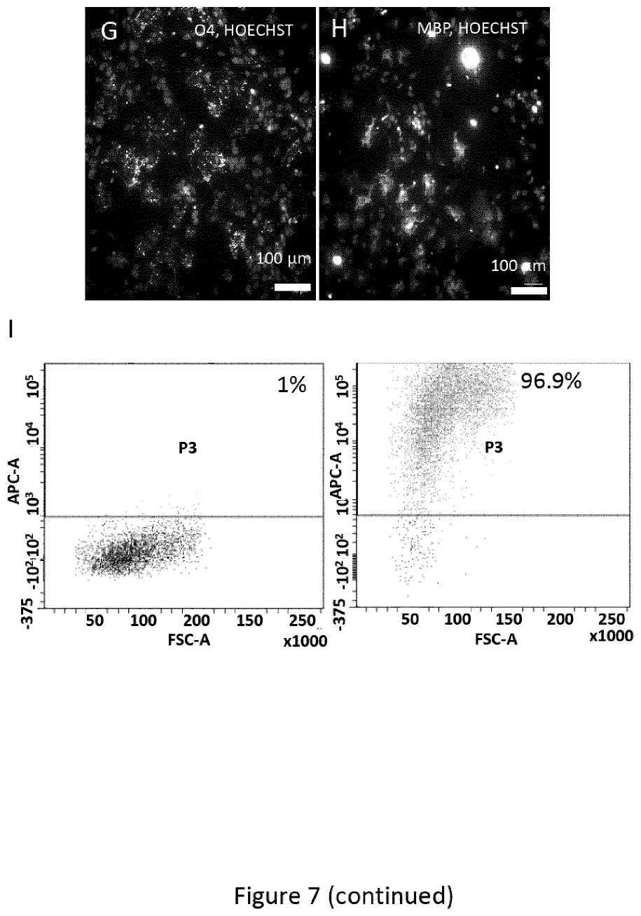

FIG. 7: Characterization of astrocyte progeny and OPCs from iSOX9 and iSOX10 iPSC

iSOX9 iPSCs were differentiated to astrocytes (panels A-E) by SOX9 transcription factor overexpression by doxycycline addition from day 11 until day 18 and maintained in astrocyte maturation medium until day 27. iSOX10 iPSCs (panels F-I) were differentiated by SOX10 transcription factor overexpression for which doxycycline was added from day 14 until day 24 and maintained in oligodendrocyte maturation medium until day 40. Cells were analyzed by RT-qPCR on the days indicated, and fluorescence immunology microscopy on day 31 for iSOX9 astrocyte progeny and day 25 for iSOX10 OPCs was done. (A): RT-qPCR analysis of iSOX9 iPSC on day −2 and astrocyte differentiated progeny on days 18 and 27 after starting differentiation, and transcript levels compared with the housekeeping gene GAPDH (data shown as delta CT). N=2 biological replicates with 3 technical replicates. Data are shown as mean±SD; One-Way ANOVA, multiple comparisons with Bonferroni correction: *: p<0.05; **: p<0.01; ***: p<0.001. (B-E) Day 27 iSOX9 astrocyte progeny was stained with antibodies against S100β, EAAT1, ALDH1L1 and GFAP, as well as Hoechst to identify the nuclei. Scale bars: 100 μM. Representative image for 33 images each for 3 wells, taken per differentiation and 2 biological replicates. (F) RT-qPCR analysis of iSOX10 iPSC on day −1 and oligodendrocyte differentiated progeny on day 12 and 24 after starting differentiation, and transcript levels compared with the housekeeping gene GAPDH (data shown as delta CT). N=2 biological replicates and 3 technical replicates. Data are shown as mean±SD; One-Way ANOVA, multiple comparisons with Bonferroni correction: p<0.05; **: p<0.01; ***: p<0.001. (G-H) Day 31 iSOX10 oligodendrocyte progeny was stained with antibodies against 04 and MBP, as well as Hoechst to identify the nuclei. Scale bars: 100 μM. Representative image for 33 images each for 3 wells, taken per differentiation and 2 biological replicates. (I) Flow cytometry analysis using a conjugated anti-04-APC antibody for staining day 24 iSOX10 OPCs and unstained day 24 iSOX10 OPCs as negative control.

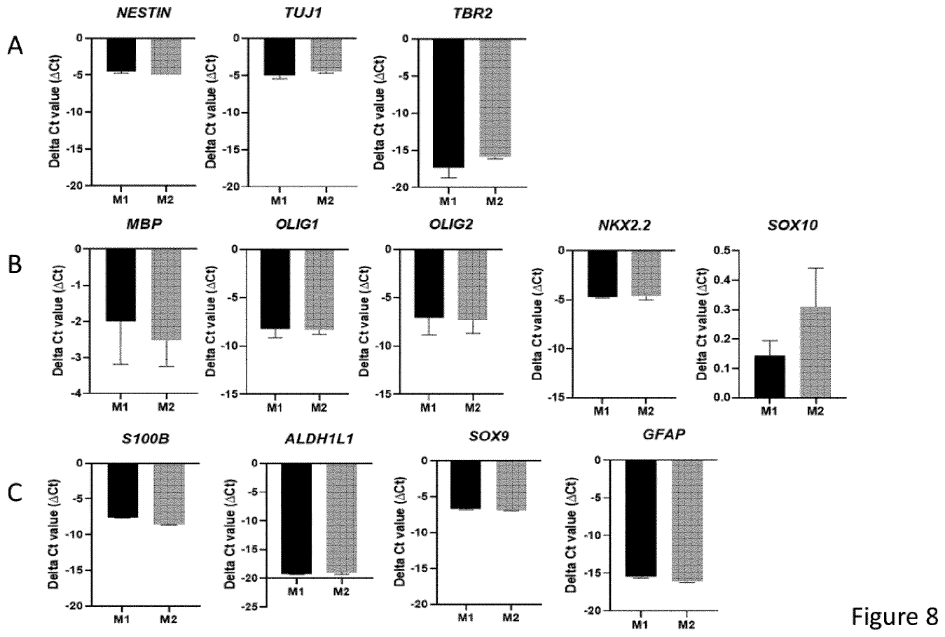

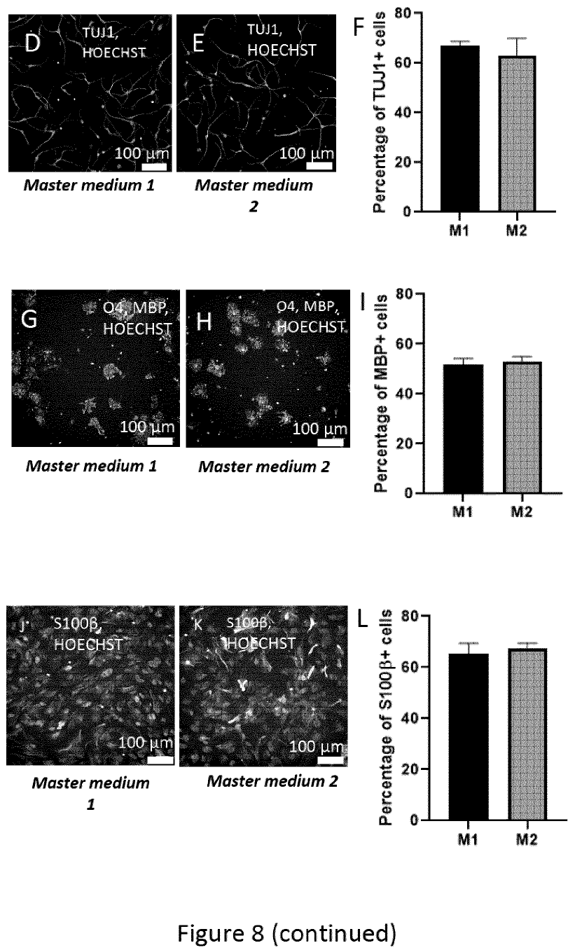

FIG. 8: Characterization of master medium composition allowing survival of neuronal progeny from iPSC, iSOX9 astrocyte progeny and iSOX10 OPCs.

SIGi001-A, iSOX9, iSOX10 iPSC lines were differentiated to cortical neuron progenitors, iSOX9-astrocyte progeny, iSOX10-OPCs using either dual-SMAD inhibition or transcription factor overexpression. Differentiated day 38 SIGi001-A NPCs, day 27 iSOX9 astrocyte progenitors and day 24 iSOX10 OPCs were cultured in M1 and M2 medium separately for 1 week. Cells were analyzed by RT-qPCR in the indicated medium after 1 week, and fluorescence microscopy on day 45 SIGi001-A NPCs, day 34 iSOX9 astrocytes and day 31 iSOX10 oligodendrocytes was done. qRT-PCR analysis of (A) neuronal differentiated progeny day 45, (B) iSOX10 oligodendrocyte progeny day 31, (C) iSOX9 astrocyte progeny day 34; in two different mediums M1 and M2, transcript levels compared with the housekeeping gene GAPDH (data shown as delta CT). N=2 biological replicates with 3 technical replicates. Data are shown as mean±SD; One-Way ANOVA, multiple comparisons with Bonferroni correction: *: p<0.05; **: p<0.01; ***: p<0.001. (D-E) Day 45 iPSC neuronal progeny was stained with an antibody against TUJ1 as well as Hoechst to identify the nuclei, in M1 and M2 mediums, (F) Quantification of percentage of TUJ1+ cells in M1 and M2 medium, (G-H) Day 31 iSOX10 oligodendrocyte progeny was stained with antibodies against MBP and 04 as well as Hoechst to identify the nuclei, in M1 and M2 medium, (I) Quantification of percentage of MBP+ cells in M1 and M2 mediums, (J-K) Day 34 iSOX9 astrocyte progeny was stained with antibodies against S100β as well as Hoechst to identify the nuclei, in M1 and M2 medium, (I) Quantification of percentage of S100β+ cells in M1 and M2 mediums. Data are shown as mean±SD. Scale bars: 100 μM. Representative image for 33 images each for 3 wells, taken per differentiation and 2 biological replicates.

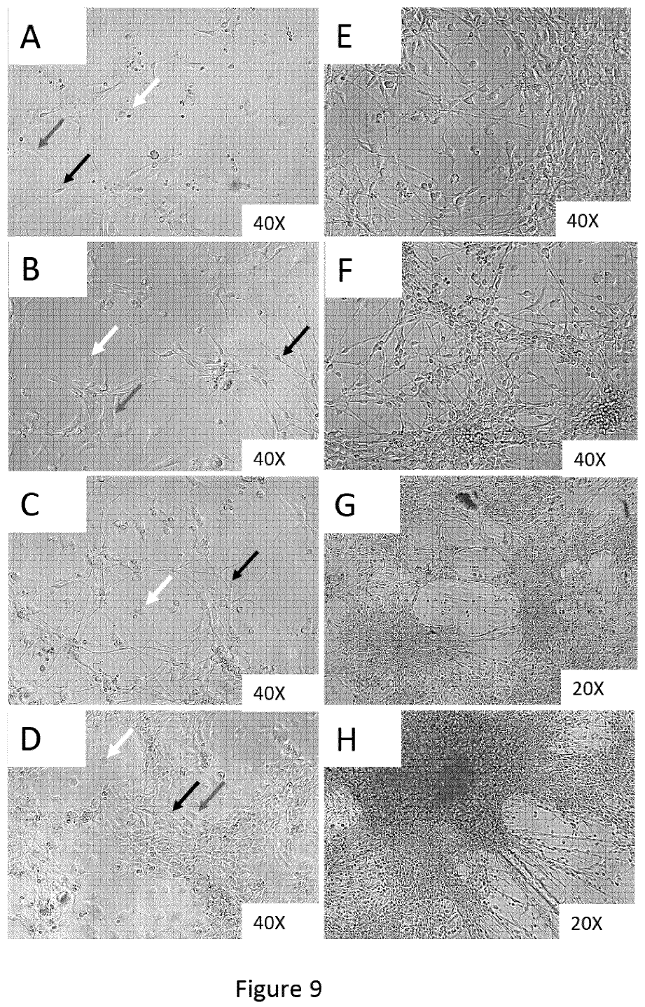

FIG. 9: Characterization of cell morphology of SIGi001-A NPCs, iSOX9 astrocyte progeny and iSOX10 OPCs in co-seeded triculture system in different cell density and culture duration.

Differentiated day 38 SIGi001-A NPCs, day 27 or day 37 iSOX9-astrocyte progenitors, and day 24 iSOX10-OPCs were co-seeded in M1 medium in equal cell ratios at different cell densities. Brightfield images were taken every week and cells were cocultured for 3 weeks in M1 medium. (A-D) Brightfield images of triculture system after 1 week in different cell densities, (A) 2,500 cells/type, (B) 5,000 cells/type, (C) 10,000 cells/type, (D) 30,000 cells/type taken at 40× magnification. White arrow in the images indicates cell morphology similar to oligodendrocytes, black arrow indicates neuronal morphology and grey arrow represent cells with astrocyte like morphology, (E-H) Brightfield images of triculture system containing 10,000 cells/type in equal ratio, taken after (E-F) two weeks and (G-H) three weeks of seeding at 40× and 20× magnification. N=2 biological replicates and 3 technical replicates. Representative image for 33 images each for 3 wells.

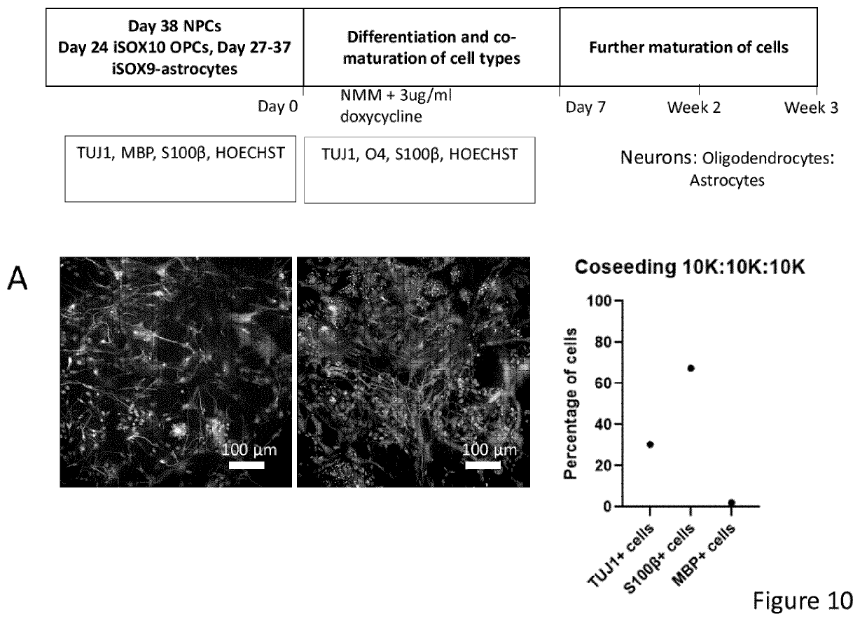

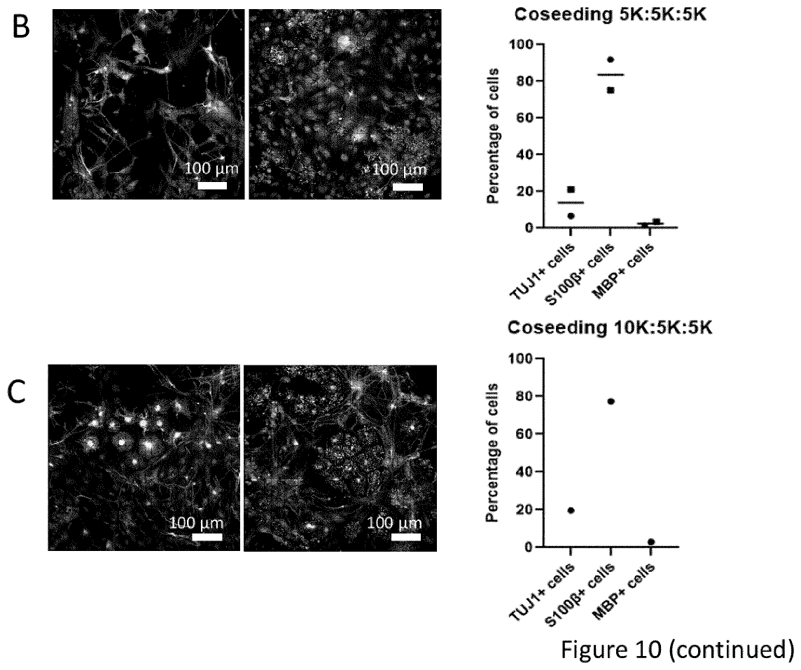

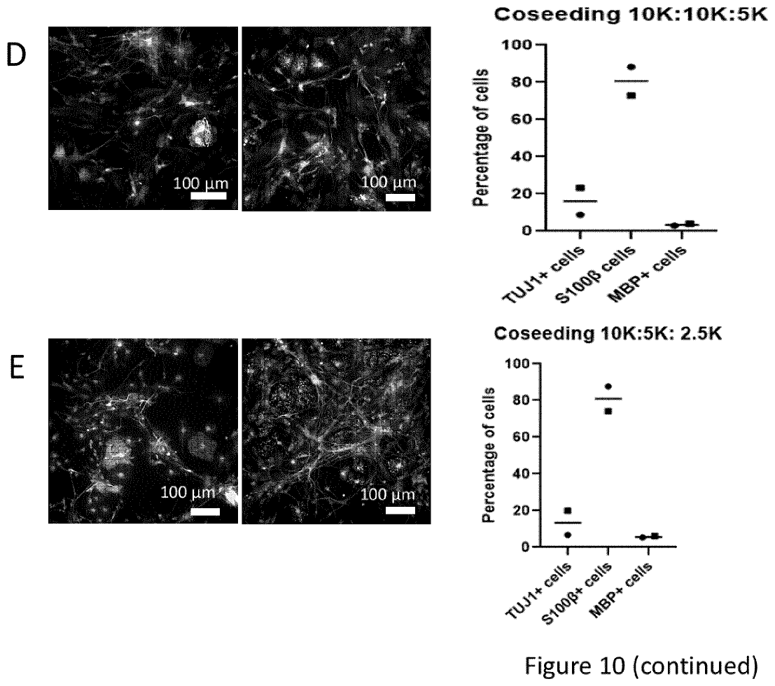

FIG. 10: Human triculture system set-up by co-seeding SIGi001-A NPCs, iSOX9 astrocyte progenitors and iSOX10 OPCs

Triculture system was set-up by co-seeding differentiated day 38 SIGi001-A NPCs, day 27 or day 37 iSOX9-astrocyte progenitors, and day 24 iSOX10-OPCs in M1 medium with 3 μg doxycycline in different cell ratios for 1 week. Cells were analysed by fluorescence immunology microscopy after 1 week of coculture. Day 38 SIGi001-A NPCs, day 27 or day 37 iSOX9 astrocyte progeny and day 24 iSOX10 OPCs plated as (A) 10,000 cells/type in equal ratio, (B) 5,000 cells/type in equal ratio, (C) 10,000 neurons, 5,000 oligodendrocytes and 5,000 astrocytes, (D) 10,000 neurons, 10,000 oligodendrocytes and 5,000 astrocytes, (E) 10,000 neurons, 5,000 oligodendrocytes and 2,500 astrocytes, were stained with TUJ1 for neurons, S100β for astrocytes, MBP and 04 for oligodendrocytes, as well as Hoechst to identify the nuclei. Black circle represents cell combination with day 27 iSOX9 astrocyte progeny and black circle indicates day 37 iSOX9 astrocyte precursors. Data are shown as mean. Scale bars: 100 μM. N=2 biological replicates and 3 technical replicates. Representative image for 33 images each for 3 wells.

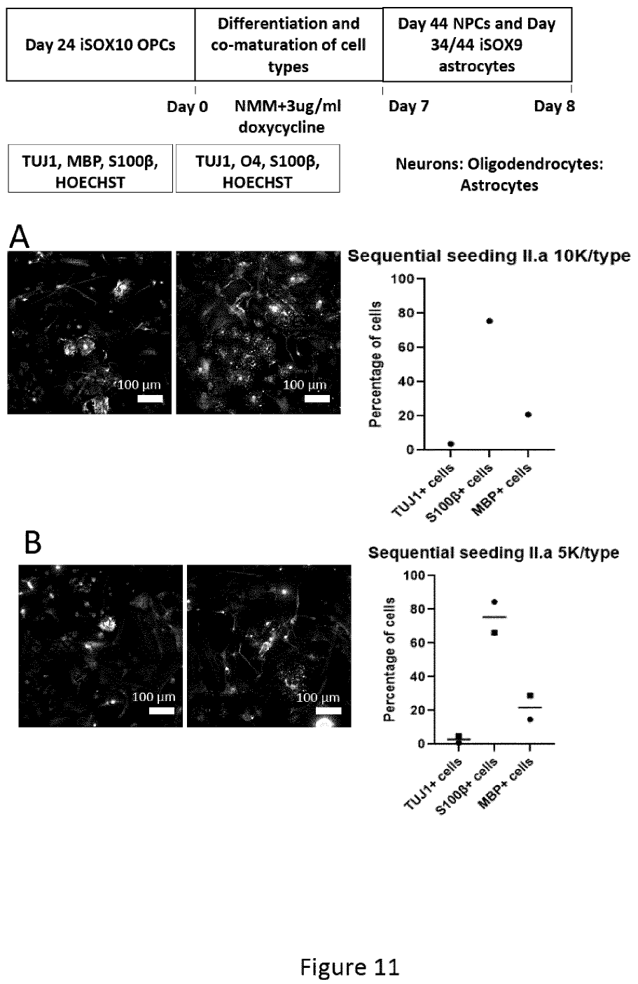

FIG. 11: Human triculture system set-up by seeding iSOX10 OPCs, followed by addition of SIGi001-A NPCs and iSOX9 astrocyte progenitors at day 7

Triculture system was set-up by sequentially seeding the day 24 iSOX10 OPCs in M1 medium with 3 μg doxycycline at first, followed by addition of day 27 or day 37 iSOX9 astrocyte progenitors and day 38 SIGi001-A NPCs at different cell ratios and densities, after 7 days. Cells were seeded in equal ratio and different cell densities and analysed by fluorescence immunology microscopy 1 day after addition of residual cells. (A-B) Day 38 neural progenitor, day 27 or day 37 iSOX9 astrocyte progeny and day 24 iSOX10 oligodendrocyte progenies plated as (A) 10,000 cells/type in equal ratio, (B) 5,000 cells/type in equal ratio and stained with TUJ1 for neurons, S100β for astrocytes, MBP and 04 for oligodendrocytes, as well as Hoechst to identify the nuclei at day 8. Black circle represents cell combination with day 27 iSOX9 astrocyte progeny and black circle indicates day 37 iSOX9 astrocyte precursors. Data are shown as mean. Scale bars: 100 μM. N=2 biological replicates and 3 technical replicates. Representative image for 33 images each for 3 wells.

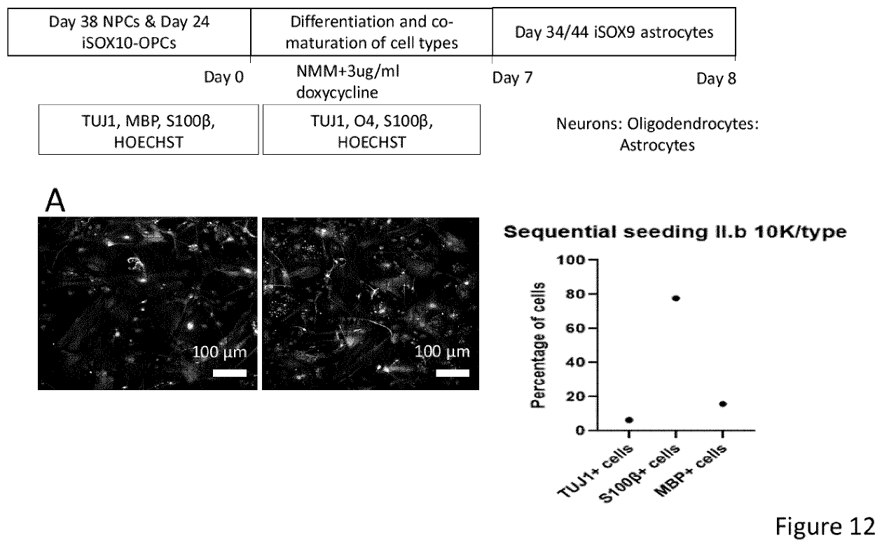

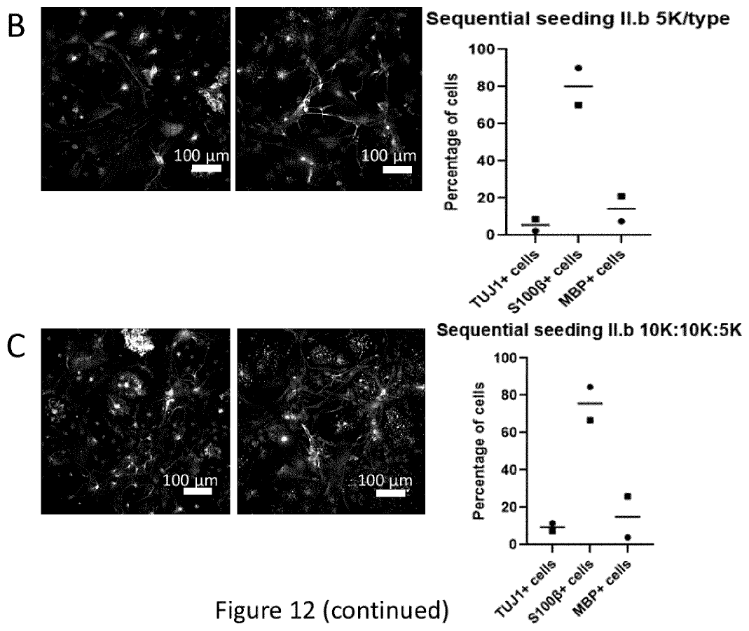

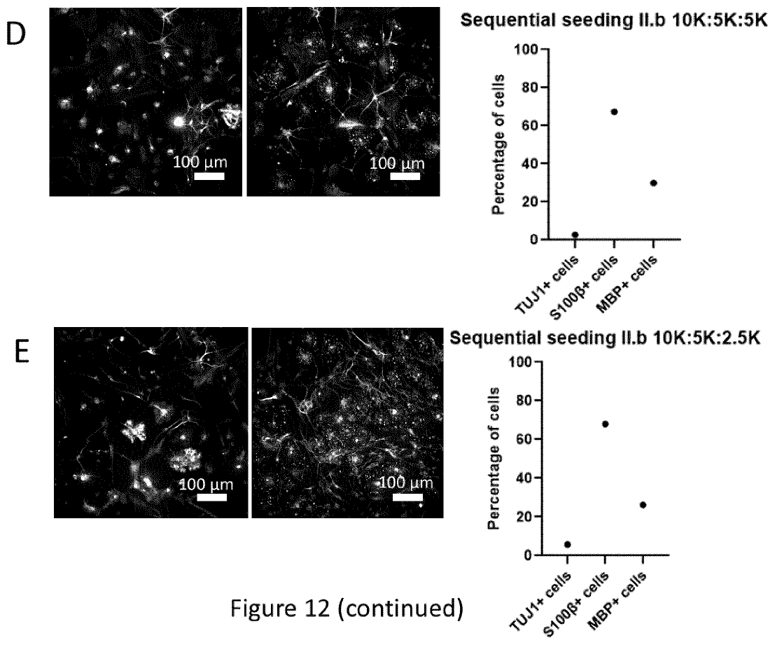

FIG. 12: Human triculture system set-up by seeding iSOX10 OPCs and SIGi001-A NPCs, followed by addition of iSOX9 astrocyte progenitors at day 7.

Triculture system was set-up by sequentially seeding the day 24 iSOX10 OPCs and day 38 NPCs in M1 media with 3 μg doxycycline at first, followed by addition of day 27 or day 37 iSOX9 astrocyte progenitors after 7 days. Cells seeded at different cell densities and ratios, were analysed by fluorescence immunology microscopy 1 day after addition of the residual cells. (A-E) Day 38 neural progenitor, day 27 or day 37 iSOX9 astrocyte progeny and day 24 iSOX10 oligodendrocyte progenies plated as (A) 10,000 cells/type in equal ratio, (B) 5,000 cells/type in equal ratio, (C) 10,000 neurons, 10,000 astrocytes and 5,000 oligodendrocytes, (D) 10,000 neurons, 5,000 astrocytes and 5,000 oligodendrocytes, (E) 10,000 neurons, 5,000 oligodendrocytes and 2,500 astrocytes, were stained with TUJ1 for neurons, S100β for astrocytes, MBP and 04 for oligodendrocytes, as well as Hoechst to identify the nuclei. Black circle represents cell combination with day 27 iSOX9 astrocyte progeny and black circle indicates day 37 iSOX9 astrocyte precursors. Data are shown as mean. Scale bars: 100 μM. N=2 biological replicates and 3 technical replicates. Representative image for 33 images each for 3 wells.

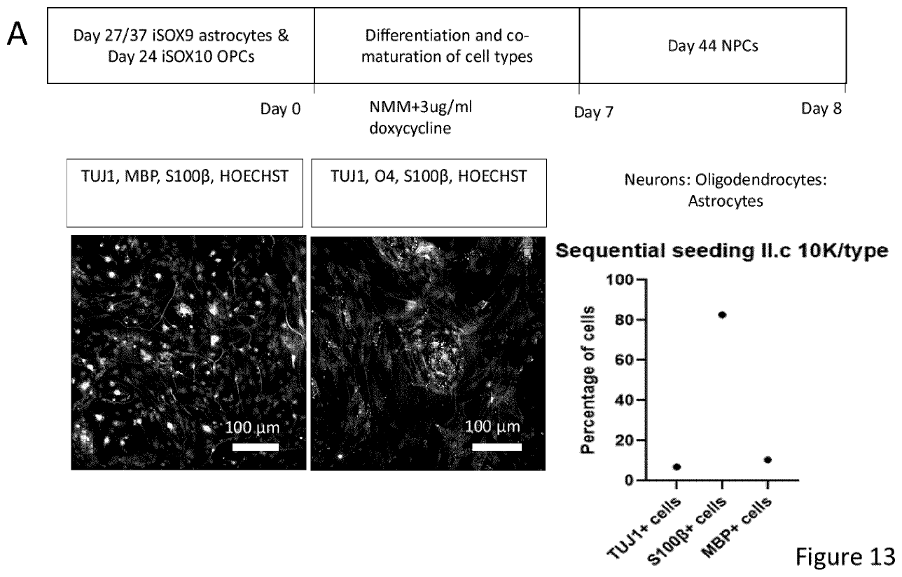

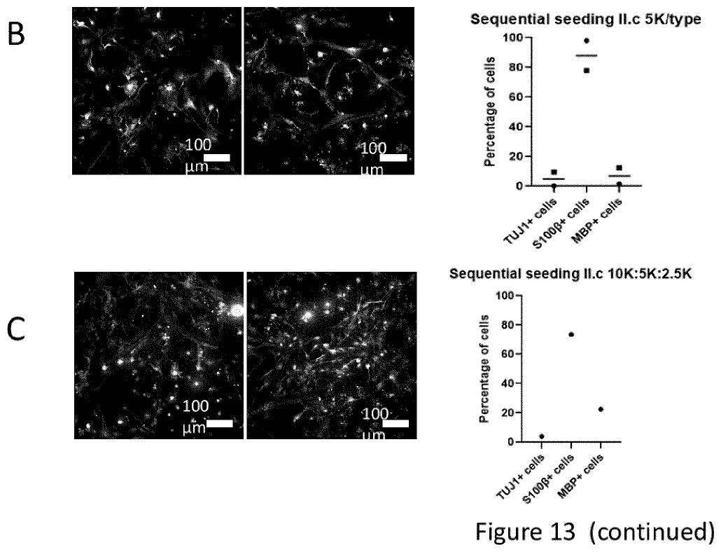

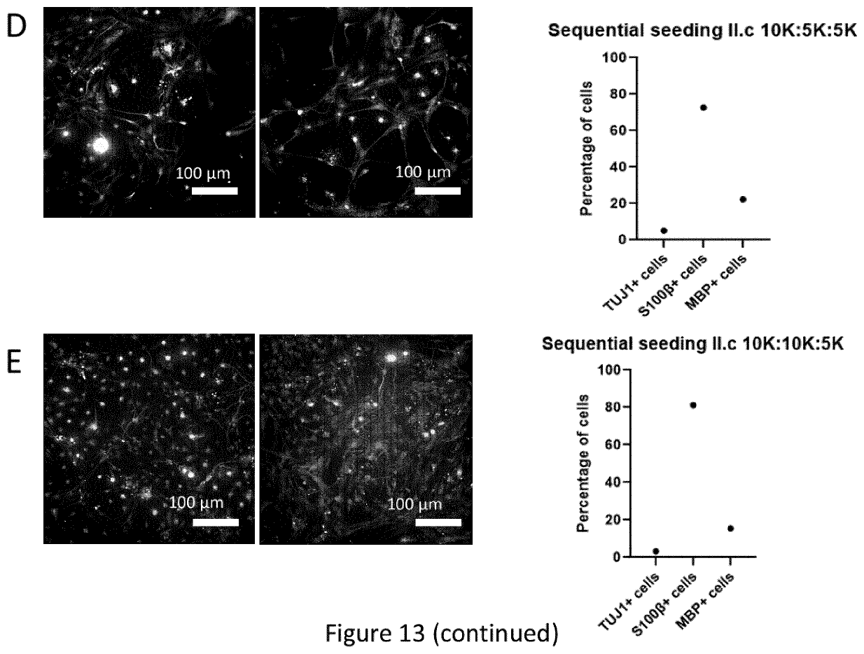

FIG. 13: Human triculture system set-up by seeding iSOX10 OPCs and iSOX9 astrocyte progenitors, followed by addition of SIGi001-A NPCs at day 7.

Triculture system was set-up by sequentially seeding the day 24 iSOX10 OPCs and day 27 or day 37 iSOX9 astrocyte progenitors in M1 media with 3 μg doxycycline at first, followed by addition of day 38 SIGi001-A NPCs after 7 days. Cells seeded at different cell densities and ratios, were analysed by fluorescence immunology microscopy 1 day after addition of residual cells. (A-E) Day 38 SIGi001-A NPCs, day 27 or day 37 iSOX9 astrocyte progeny and day 24 iSOX10 OPCs plated as (A) 10,000 cells/type in equal ratio, (B) 5,000 cells/type in equal ratio, (C) 10,000 neurons, 5,000 oligodendrocytes and 2,500 astrocytes, (D) 10,000 neurons, 5,000 oligodendrocytes and 5,000 astrocytes, (E) 10,000 neurons, 10,000 oligodendrocytes and 5,000 astrocytes, were stained with TUJ1 for neurons, S100β for astrocytes, MBP and 04 for oligodendrocytes, as well as Hoechst to identify the nuclei. Black circle represents cell combination with day 27 iSOX9 astrocyte progeny and black circle indicates day 37 iSOX9 astrocyte precursors. Data are shown as mean. Scale bars: 100 μM. N=2 biological replicates and 3 technical replicates. Representative image for 33 images each for 3 wells.

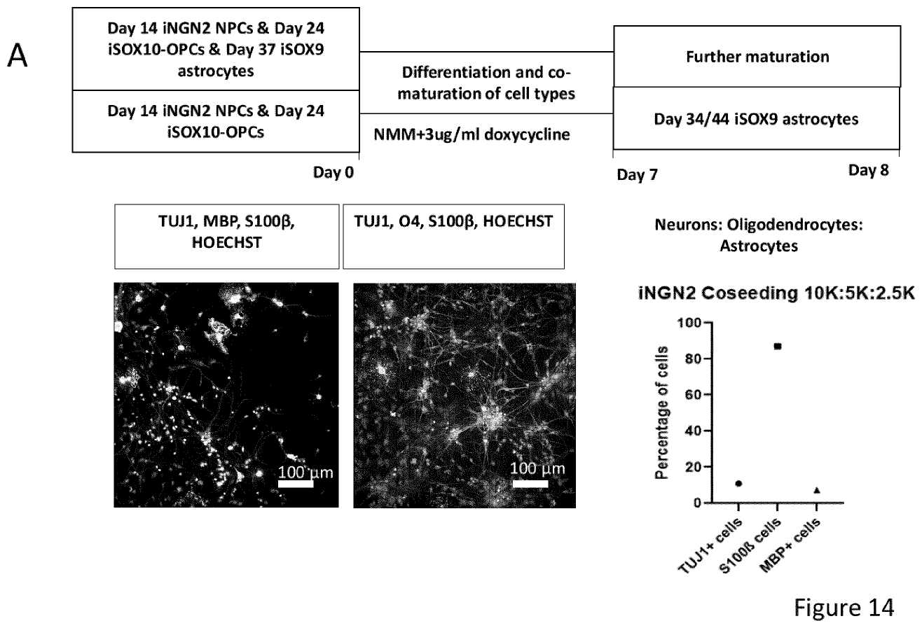

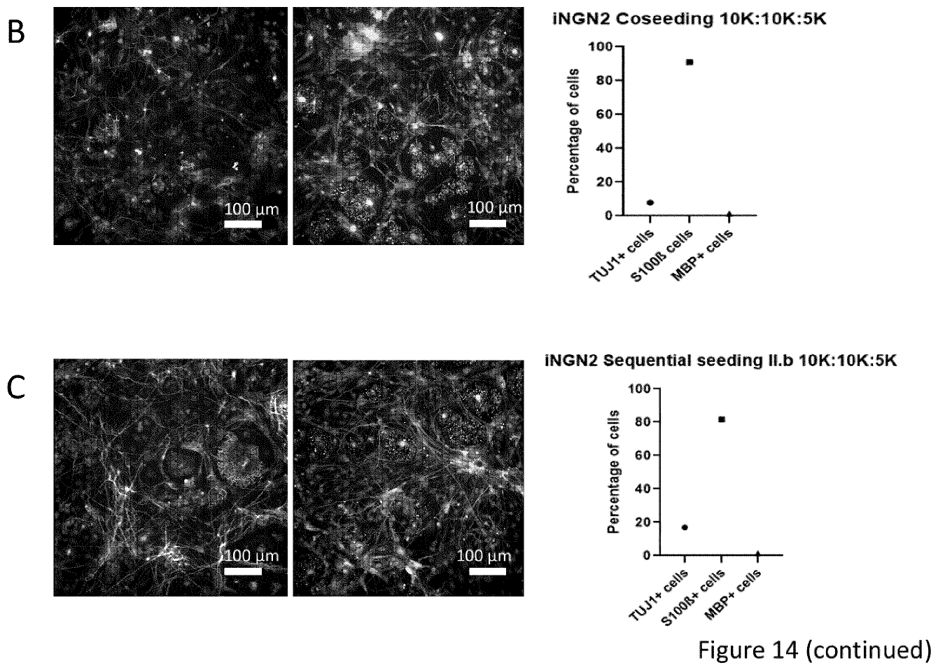

FIG. 14: Human triculture system containing BIONi010-C iNGN2 NPCs, iSOX10 OPCs and iSOX9 astrocyte progenitors by co-seeding and sequential cell seeding.

Triculture system was set-up by both co-seeding (panels A and B) and sequential seeding (panel C) of day 14 BIONi010-C iNGN2 neuron progeny, day 24 iSOX10 OPCs and day 27 or 37 iSOX9 astrocyte progenitors in M1 media with 3 μg doxycycline. (A-B) Day 14 BIONi010-C iNGN2 NPCs, day 27 or day 37 iSOX9 astrocyte progeny and day 24 iSOX10 OPCs were co-seeded as, (A) 10,000 BIONi010-C iNGN2 NPCs, 5,000 iSOX10 OPCs and 5000 iSOX9 astrocyte progenies, (B) 10,000 BIONi010-C iNGN2 NPCs, 10,000 iSOX10 OPCs and 5,000 iSOX9 astrocyte progenies, and stained with TUJ1 for neurons, S100β for astrocytes, MBP and 04 for oligodendrocytes, as well as Hoechst to identify the nuclei. (C) 10,000 BIONi010-C day 14 iNGN2 NPCs, 10,000 day 24 iSOX10 OPCs were seeded in M1 medium with 3 μg doxycycline, followed by addition of 5,000 day 37 iSOX9 astrocyte precursors after 1 week. Black circle represents cell combination with day 27 iSOX9 astrocyte progeny and black circle indicates day 37 iSOX9 astrocyte precursors. Data are shown as mean. Scale bars: 100 μM. N=2 biological replicates and 3 technical replicates. Representative image for 33 images each for 3 wells.

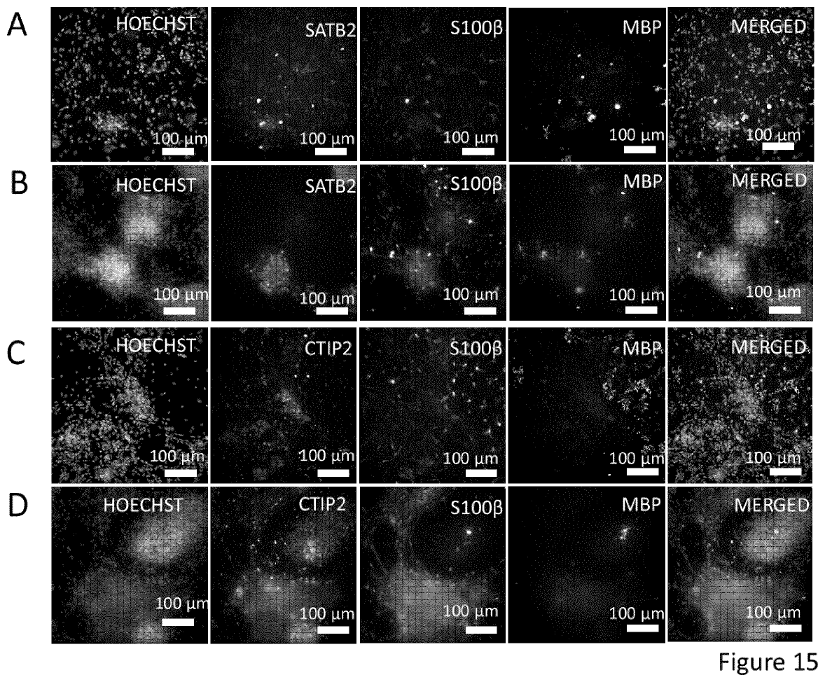

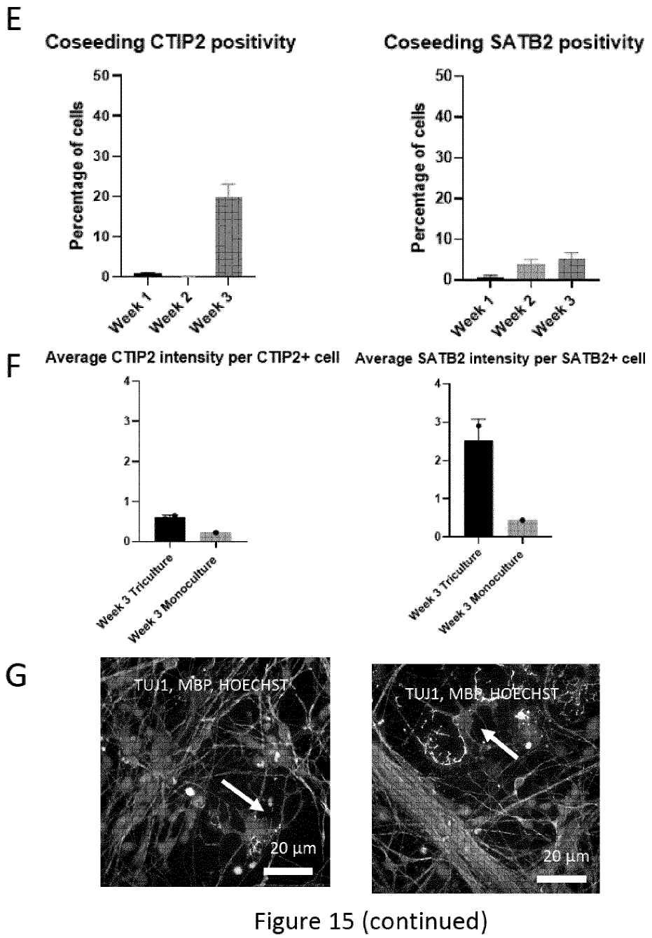

FIG. 15: Maturation of cortical neurons in human triculture system

SIGi001-A NPCs were used to set up monoculture and triculture system by co-seeding differentiated day 38 neuronal progeny, day 27 or day 37 iSOX9-astrocyte progenitors, and day 24 iSOX10-OPCs in equal cell ratio, in M1 medium with 3 μg doxycycline for 1 week, followed by medium change with only M1 medium till week 3. Cells were analysed by fluorescence immunology microscopy after 7 days for 3 weeks. (A-D) Neural progenitors stained with SATB2 and CTIP2, astrocytes stained with S100β and oligodendrocytes with MBP after 2 weeks (A and C) and 3 weeks (B and D) of triculture. Scale bars: 100 μM. N=2 biological replicates and 3 technical replicates. Representative image for 33 images each for 3 wells, taken per differentiation and 2 biological replicates. (E) Quantification of percentage of CTIP2+ and SATB2+ cells after 2 and 3 weeks of triculture, (F) Quantification of CTIP2 and SATB2 positivity per cell in the third week of triculture and monoculture. (G) Neural progenitor stained with TUJ1, and oligodendrocytes stained with MBP, after 3 weeks of triculture. Scale bars: 20 μM. Data are shown as mean±SD. N=1 biological replicate and 3 technical replicates.

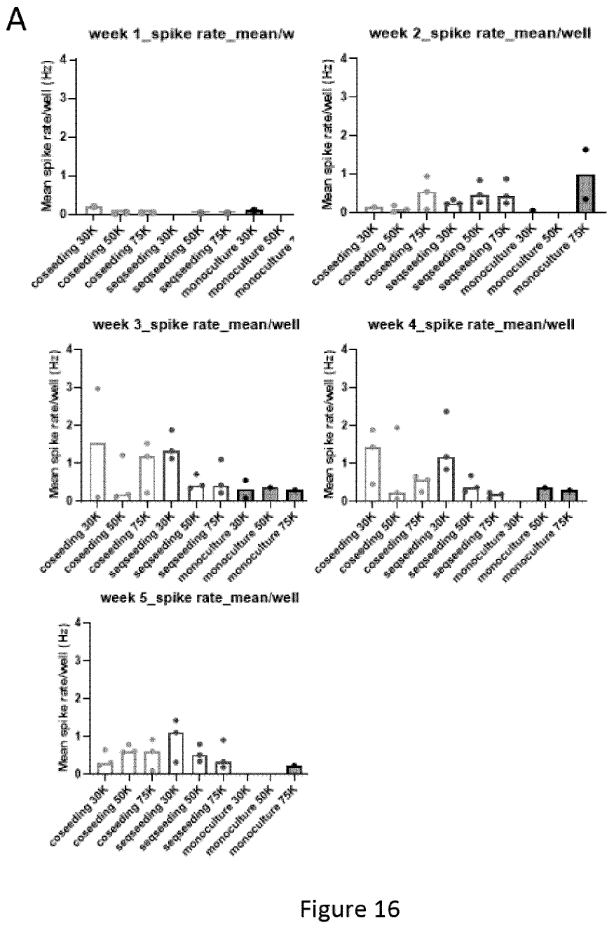

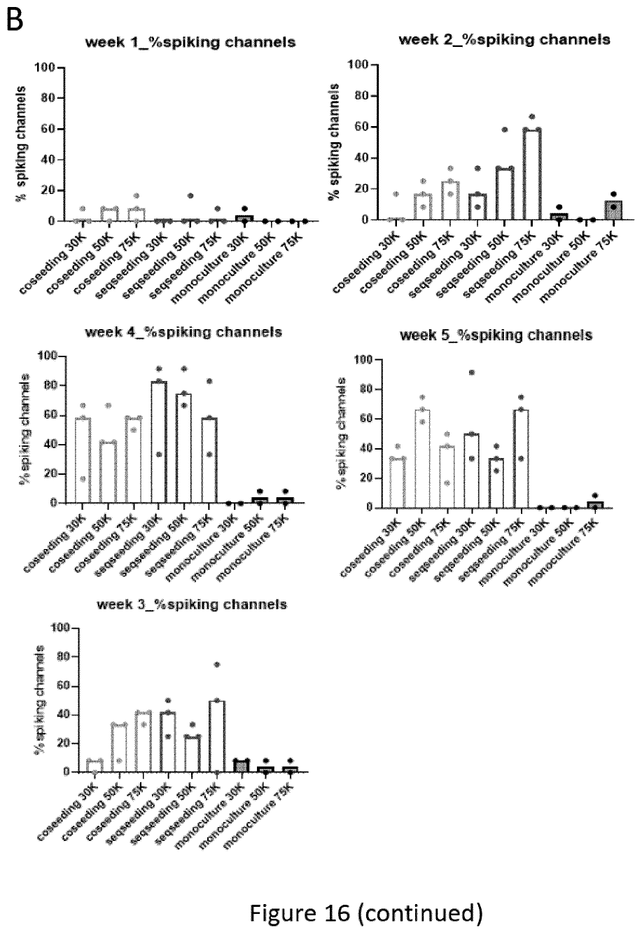

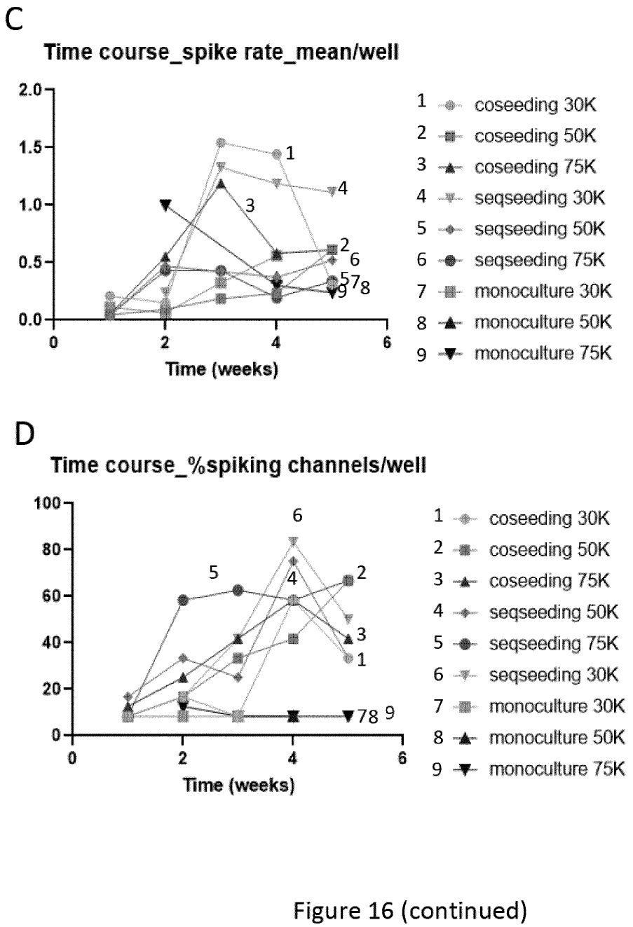

FIG. 16: Maturation of BIONi010-C iNGN2 neurons in triculture system in terms of neuronal spikes

BIONi010-C iNGN2 iPSC derived neural progenitors were used to set-up triculture system on MEA system for 5 weeks by co-seeding and sequential seeding (neuron-oligodendrocytes plating, followed by astrocyte addition after 1 week) the differentiated day 4 iNGN2 neuron precursors, day 46 iSOX9 astrocytes and day 24 iSOX10 OPCs (neurons: 2 and astrocytes and oligodendrocytes: 1 ration), in M1 medium supplemented by 3 μg doxycycline for the first week and only M1 medium for the following 4 weeks. Three cell densities were used, 30,000 neurons and glia, 50,000 neurons and glia, and 75,000 neurons and glia. MEA recording was done every week for 5 weeks in total. (A) Mean spike rate per well for co-seeded, sequentially seeded triculture and neuron monoculture for 5 weeks, (B) Percentage of active spiking channels per well for co-seeded and sequentially seeded triculture and neuron monoculture for 5 weeks, (C-D) Time-course analysis of spike rate and active spiking channels in co-seeded and sequentially seeded triculture and monoculture for 5 weeks. Data are shown as median. N=1 biological replicate and 3 technical replicates for tricultures and 2 technical replicates for neuron monoculture.

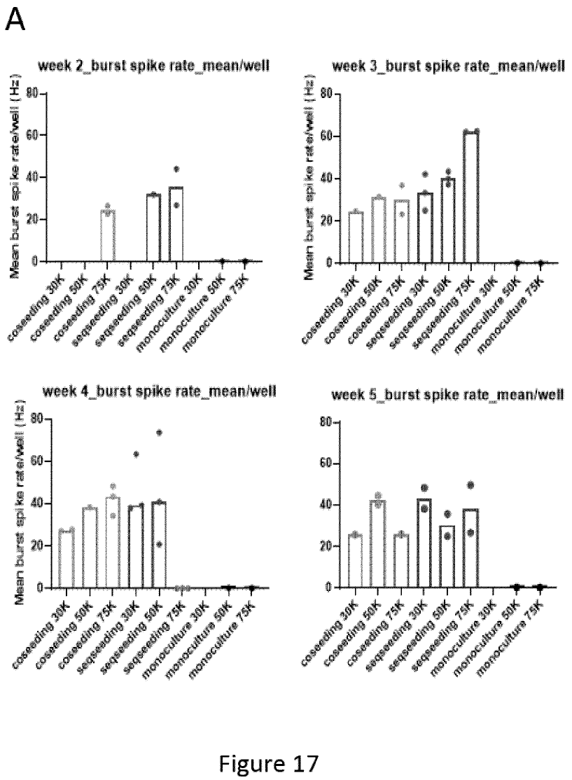

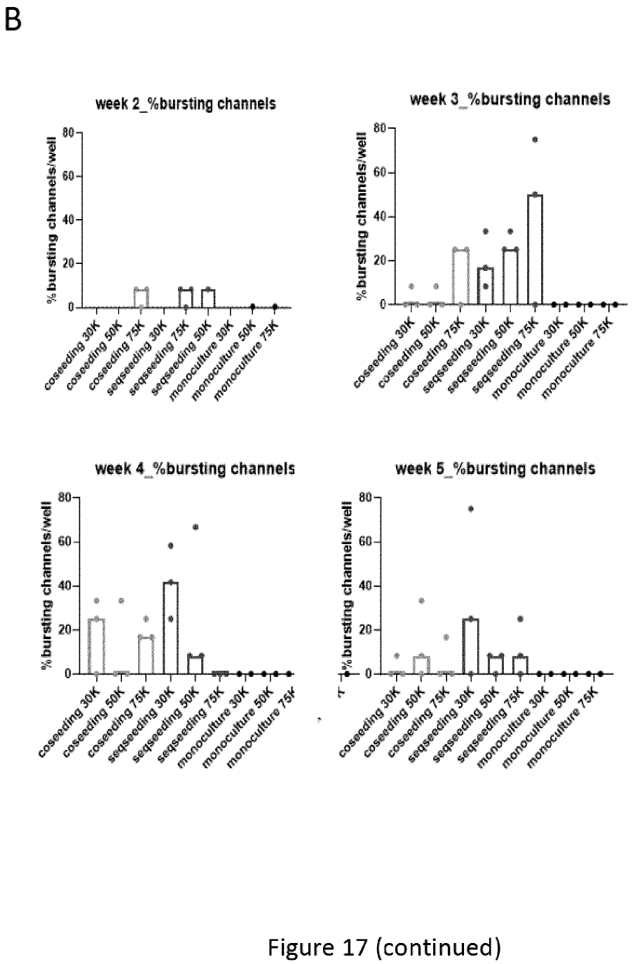

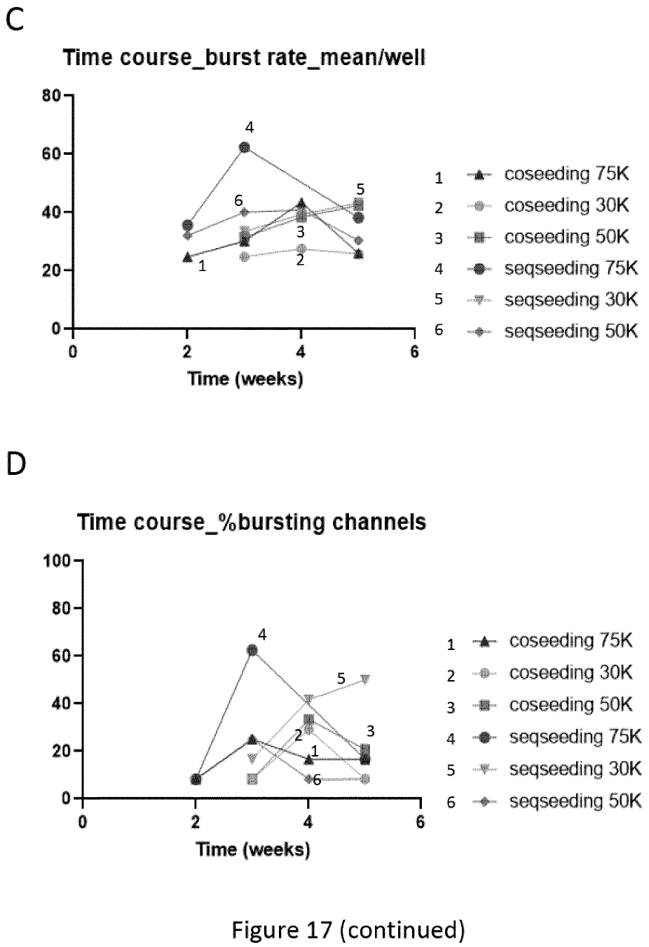

FIG. 17: Maturation of BIONi010-C iNGN2 neurons in triculture system in terms of neuronal burst activity

BIONi010-C iNGN2 iPSC derived neural progenitors were used to set-up triculture system on MEA system for 5 weeks by co-seeding and sequential seeding (neuron-oligodendrocytes plating, followed by astrocyte addition after 1 week) the differentiated day 4 BIONi010-C iNGN2 neuron precursors, day 46 iSOX9 astrocytes and day 24 iSOX10 OPCs (neurons:2, astrocytes and oligodendrocytes each:1), in M1 medium supplemented by 3 μg doxycycline for first week and only M1 medium for the following 4 weeks. Three cell densities were used, 30,000 neurons and glia, 50,000 neurons and glia, and 75,000 neurons and glia, with equal number of neurons in monocultures. MEA recording was done after every week, for 5 weeks in total. (A) Burst rate per mean per well for co-seeded, sequentially seeded triculture and neuron monoculture for 5 weeks, (B) Percentage of active bursting channels per well for co-seeded and sequentially seeded triculture and neuron monoculture for 5 weeks, (C-D) Time-course analysis of burst rate and active bursting channels in co-seeded and sequentially seeded triculture and monoculture for 5 weeks. Data are shown as median. N=1 biological replicate and 3 technical replicates for tricultures and 2 technical replicates for neuron monoculture.

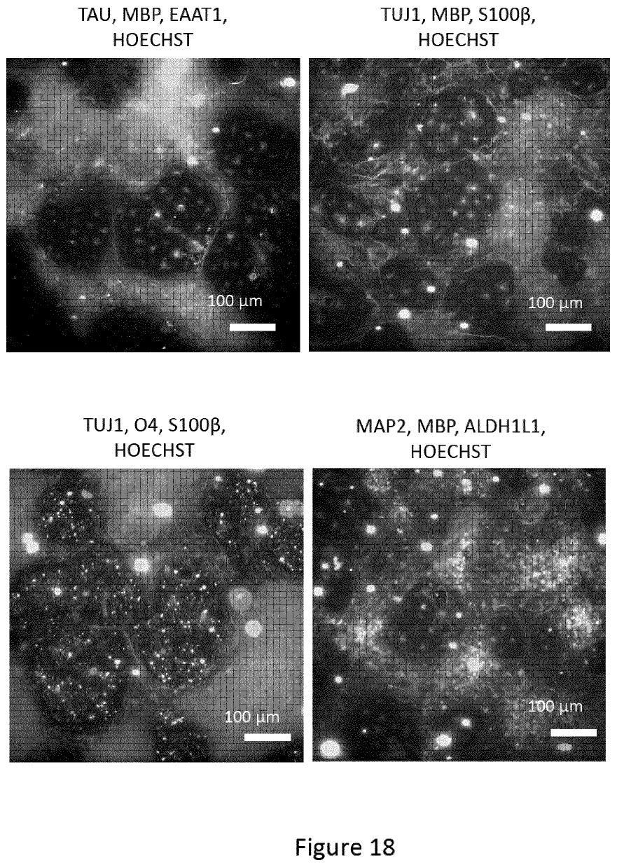

FIG. 18 Antibody combination optimization for characterizing and quantifying BIONi010-C iNGN2 and cortical neurons, iSOX9 astrocyte precursors, and iSOX10 OPCs in human triculture system.

Triculture system was set-up by co-seeding differentiated day 38 SIGi001-A NPCs, day 27 or day 37 iSOX9-astrocyte progenitors, and day 24 iSOX10-OPCs in M1 medium with 3 μg doxycycline in equal cell ratios and 30,000 cells/type cell density for 1 week. Cells were analysed by fluorescence microscopy with different antibody combinations after 1 week of coculture. Neuronal progeny was stained with TUJ1 and MAP2, astrocyte progenitors stained with S100β, EAAT1 and ALDH1L1, oligodendrocyte precursors stained with MBP and 04, along with Hoechst to identify the nuclei. Scale bars: 100 μM. N=2 biological replicates and 3 technical replicates. Representative image for 33 images each for 3 wells, taken per differentiation and 2 biological replicates.

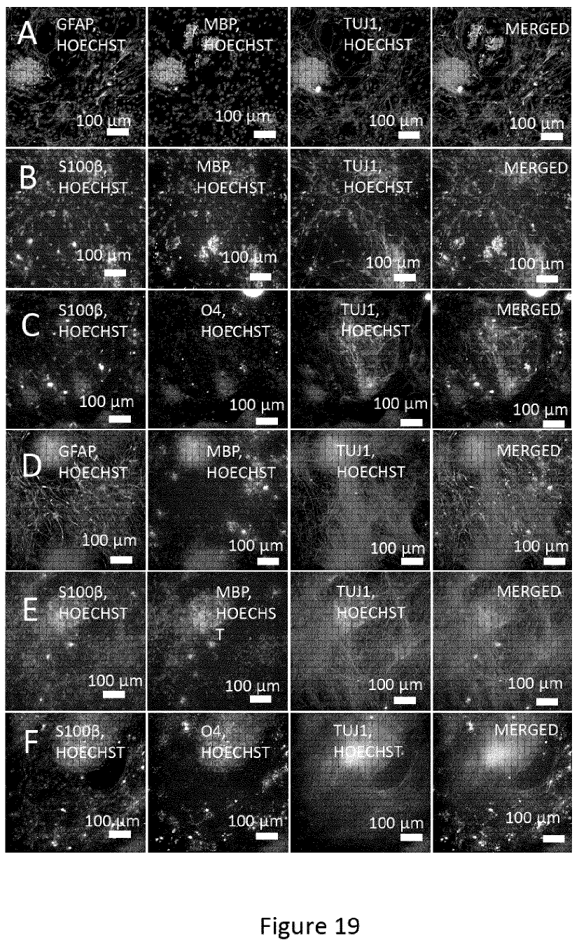

FIG. 19: Antibody combinations for week 2 and 3 triculture system containing cortical neurons, iSOX9 astrocytes, and iSOX10 oligodendrocytes.

SIGi001-A iPSCs derived neural progenitors were used to set up monoculture and triculture system by co-seeding differentiated day 38 neuronal progeny, day 27 or day 37 iSOX9-astrocyte progenitors, and day 24 iSOX10-OPCs in equal cell ratio, in M1 medium with 3 μg doxycycline for 1 week, followed by medium change with only M1 medium till week 3. Cells were analysed by fluorescence microscopy after 7 days for 3 weeks. (A-F) Neurons were stained with TUJ1, oligodendrocytes stained with MBP and 04, and astrocytes stained with S100β and GFAP after 2 weeks (A-C) and 3 weeks (D-F) of triculture. Scale bars: 100 μM. N=1 biological replicate with 3 technical replicates. Representative image for 33 images each for 3 wells

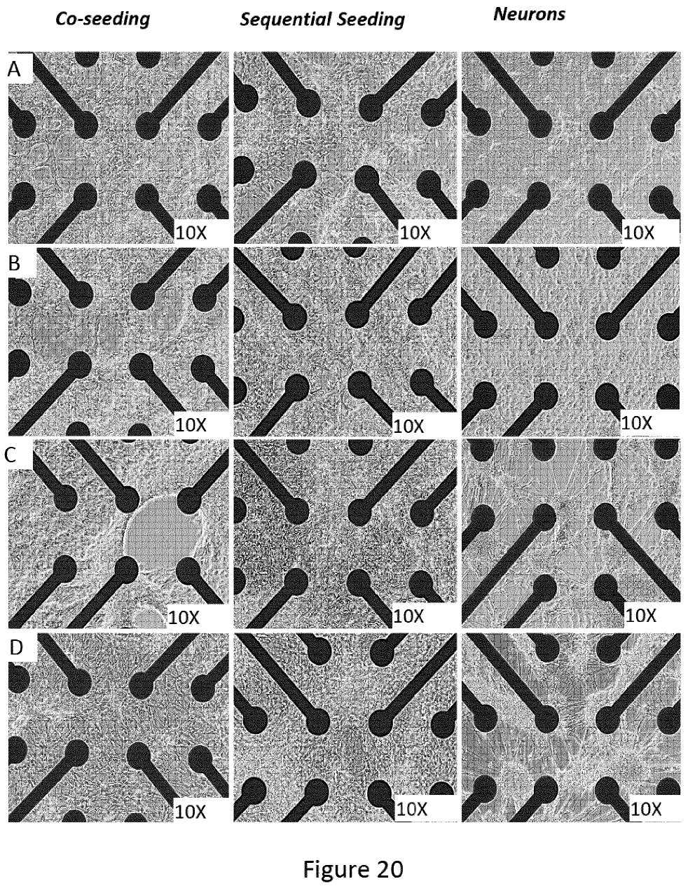

FIG. 20: Characterization of cell morphology of BIONi010-C iNGN2 NPCs, iSOX9 astrocyte progeny and iSOX10 OPCs in co-seeded, sequential seeded triculture and monoculture system on MEA in different cell density.

BIONi010-C iNGN2 iPSC derived neural progenitors were used to set-up triculture system on MEA system for 5 weeks by co-seeding and sequential seeding (neuron-oligodendrocytes plating, followed by astrocyte addition after 1 week) the differentiated day 4 BIONi010-C iNGN2 neuron precursors, day 46 iSOX9 astrocytes and day 24 iSOX10 OPCs in twice the number of neurons as astrocytes and oligodendrocytes, in M1 medium supplemented by 3 μg doxycycline for first week and only M1 medium for the following 4 weeks. Three cell densities have been used, 30,000 neurons and glia, 50,000 neurons and glia, and 75,000 neurons and glia, with equal numbers of neurons in monocultures. MEA recording was done after every week, for 5 weeks in total. (A-D) Brightfield images taken for four weeks for co-seeded, sequential seeded triculture and neuron monocultures at 10× magnification. N=1 biological replicate with 3 technical replicates for tricultures and 2 technical replicates for neuron monoculture.

Neurons are divided into sub-categories based on their function, neurotransmitter secretion, localization, and morphology. They can be categorized as sensory neurons involved in receiving information, motor neurons that transmits signal from the brain to target site, and interneurons that acts as a junction for motor and sensory neurons.

With respect to neurotransmitter released, neurons can also be classified as:
1. Excitatory neurons releasing excitatory neurotransmitters like acetylcholine, glutamate, epinephrine, nor-epinephrine, and histamine
2. Inhibitory neurons releasing repressive neurotransmitters like serotonin and gamma-aminobutyric acid (GABA)
3. Dopaminergic neurons that have both excitatory and inhibitory functions in human brain.

The main role of neurons is to transmit impulses to other neurons, muscle, or gland cells. The neurons are generated by multipotent neural progenitor cells, which are also capable of generating astrocytes, and oligodendrocytes. The differentiation of neural progenitor cells consists of a neural rosette stage, a radial alignment of columnar cells which is also considered as a developmental signature of neural progenitor cells. Also, half of the total neuron population in found in cerebral cortex, the largest component of a mammalian brain. This cortical neuron population is organized in six different cortical layers having distinct types and density of neuron cell bodies. These layers comprise two types of neurons, pyramidal and non-pyramidal neurons classified on the basis of the shape of their soma. Pyramidal neurons connect distant targets in other areas of cortex and brain and makes up 75% of total cortical neurons. They are also referred to as excitatory cortical neurons due to their property of utilizing glutamate neurotransmitter.

In contrast therewith, non-pyramidal cells possess shorter axons and act as interneurons to connect local targets. These cells are inhibitory cortical neurons, which secretes neurotransmitter GABA and take part in tangential migration during development of cerebral cortex.

Neurons express different markers based on their developmental stage, cell components, and neurotransmitter release. The highly compartmentalized structure of neurons provides several marker expressions to differentiate them from other cell types in CNS. For example, TUJ1 is Neuron-specific Class III β-tubulin marker, Microtubule-associated protein 2 (MAP-2) is a cytoskeletal protein marker, and Neuronal Nuclei antigen (NeuN) or FOX-3 is a nuclear protein marker that helps in neuron specific labelling [Kole et al. Cell death & disease 4, e689-e689 (2013)].

Glial cells are the second group of cells in CNS that regulate neuronal functions by providing support and protection. They maintain homeostasis and form myelin sheaths around axons for efficient nerve impulse conduction. In addition, glial cells are responsible for neuronal growth support, blood-brain barrier formation and control, brain metabolism and homeostasis regulation. Two major glial cell types are present in the CNS (neuroectodermal in origin), namely astrocytes and oligodendrocytes.

Astrocytes are known as molecular regulators that secrete growth factors, neurotransmitters, and metabolic mediators, and thus support synaptogenesis, synaptic connectivity, and neuron growth.

On the other hand, oligodendrocytes physically protect and increase conductivity by insulating axons with myelin sheaths. They also provide metabolic support to axons by transferring metabolites, like lactate or pyruvate, to fuel axonal energy production.

Other glial cell types include microglia (which are however mesodermal), ependymal cells (in CNS), satellite cells (also called amphicytes), and Schwann cells (in PNS). These cells play important roles in the production of cerebrospinal fluid, myelin sheath formation and provide nutrition and support to neurons.

Astrocytes are star-shaped cells in the brain that are heterogenous in morphology, functionality, location, and origin of development. These cells can be classified as protoplasmic, interlaminar, polarized and fibrous astrocytes. Protoplasmic astrocytes are found in grey matter and fibrous astrocytes in white matter. Technologies like single cell RNA sequencing and spatial transcriptomics has been used to demonstrate inter- and intra-regional heterogeneity of astrocytes to understand their distinct morphologies and physiologies across human brain cortex. Due to their extensive diversity, characterization of these cells remains challenging. One of the most common markers used for astrocyte identification is an intermediate filament protein, glial fibrillary acidic protein (GFAP). Importantly, astrocyte maturation is an intricate process that is not fully uncovered and known to have overlapping protein expression. Roybon et al. constructed the timeline for appearance and disappearance of known astrocytic markers based in rat and human spinal cord astrocytes. However, the time-dependent development of different astrocyte classes varies in different brain regions [Roybon et al. Cell Rep. 4, 1035-1048 (2013)]. Nonetheless, expression of proteins like Vimentin, S100 calcium-binding protein beta (S100β), cell-surface glycoprotein CD44 are used to identify astrocyte progenitor cells. Excitatory amino acid 1 and 2 (EAAT1 and EAAT2), aquaporin 4 (AQP4), glutamine synthetase (GS), and aldehyde dehydrogenase 1 family member (ALDH1L1) expression are markers for mature astrocytes [Zhang et al. Biomed Res. Int. 2019, (2019) 9605265].

Astrocytes are important for healthy CNS functioning. They act as a crucial CNS component by supporting neuronal metabolism, blood-brain barrier function, synaptogenesis and uptake and excretion of neurotransmitters and ions. Several receptors are common in both astrocytes and neurons; therefore, astrocytes can also be affected by neuron-derived neurotransmitters. Hasel et al. demonstrated the role of neurons exerted on astrocyte development and maturation, and showed the involvement of neuron-astrocyte interactions like NOTCH signaling in maturation and neurotransmitter uptake activity of astrocytes [Hasel et al. Nat. Commun. 8, 1-18 (2017)]. Astrocytes also maintain the level of neuron-supporting molecules like glutamate, by controlling its excretion through glutamate transporters EAAT1 and EAAT2. They contribute significantly to the tripartite synapses, that involves the directional cellular crosstalk between pre-/post-synaptic neurons and astrocytes [Farhy-Tselnicker & Allen Neural Development 13, 1-12 (2018)]. In addition, astrocytes promote synaptogenesis and regulate the release of gliotransmitters and proteins that can affect neuron excitability. Besides regulating synapse activity, astrocytes also serve as an ion modulator. Astrocytes have variable mechanisms to import and export ions by several types of channels, like the Na+/H+ exchanger, the bicarbonate transporters, the monocarboxylic acid transporter and many others. Other functions of astrocytes include regulation of glucose and oxygen to sustain neuronal function and viability, and tissue repair after brain injury.

Oligodendrocytes or oligodendroglia, account for 3% of brain tissue. They are large glial cells produced from multipotent neural stem cells in the embryonic neural tube during neurodevelopment. Oligodendrocytes are small-sized cells with fine cytoplasmic extensions filled with granules and nuclei comprising dense chromatin. The major role of oligodendrocytes is myelination of neurons, that in turn regulates the nerve impulse conduction. Based on features of the myelination process, oligodendrocytes are classified in four major types: (1) Type 1 oligodendrocytes ensheath smaller diameter axons with a large number of myelin segments in different orientation; (2) type 2 possess the same characteristics with parallel myelin segments; (3) type 3 myelinates less number of axons with larger diameter; and (4) type 4 cells are present opposed to a single very large axon similar to Schwann cells in PNS. The oligodendrocyte precursors are characterized by expression of three markers: cyclic-nucleotide 3′-phosphodiesterase (CNPase) and the cell surface markers 04 and 01. The CNPase enzyme has two isoforms, large and small, expressed in oligodendrocyte precursors and myelinating oligodendrocytes, respectively. Cell surface marker 01 is mainly seen in pre-myelinating oligodendrocytes and 04 in late oligodendrocyte precursors. Mature myelinating oligodendrocytes express cell lineage specific myelin basic protein (MBP), transmembrane protein PLP, myelin associated glycoprotein (MAG), membrane marker galactocerebroside (GalC) and surface marker myelin-oligodendrocyte glycoprotein (MOG). OLIG1 and OLIG2 are transcription factors that are expressed throughout the oligodendroglial lineage. They are important in oligodendrocyte precursor differentiation and myelination.

The main functions of oligodendrocytes in the CNS are axon myelination during neurodevelopment, adaptive myelination in adulthood and remyelination on CNS injury. The myelin sheath wraps around axons with larger diameter in a concentric fashion and permits rapid nerve conduction. It is an electric insulator that assists in membrane potential conduction with the help of sodium channels at intermittent interruptions in the myelin sheath, called nodes of Ranvier. The process of nerve impulse conduction through myelin is known as saltatory conduction and it requires less energy due to low capacitance of myelin sheath, and therefore enables faster nerve impulse conduction than in non-myelinated axons. Myelination itself is a regulated process governed by numerous factors like calcium activity in oligodendrocytes and neuronal activity. Oligodendrocytes supports neurons metabolically by generating lactic acid, which is transferred to axons to permit ATP production. The lactic acid is cycled with the help of lactate transporters MCT1, MCT2 and MCT4 in brain and MCT1 expression is detected in oligodendrocytes. The role of oligodendrocytes in regulating neuron metabolism is also supported by the presence of several glycolytic and Krebs cycle enzymes like succinate dehydrogenase and fumarase in oligodendrocytes, that promotes glucose and ATP production, and thus supports neuron growth, survival and synaptogenesis Besides myelination, oligodendrocytes are also involved in immunomodulation. Oligodendrocyte precursors express cytokine receptors and evaluate their microenvironment through fine filopodia extensions. They also behave similar to microglia in terms of migration to injury site and cross-presenting antigens to CD8+ T cells in vitro and in vivo when interacted with interferon gamma.

Ependymal cells are another type of neuron-supporting cell that forms an epithelial lining around the brain ventricles and central canal of the spinal cord. The structure of these cells is similar to mucosal epithelial cells with tentacle-like extensions at basal surface and cilia and microvilli at apical surface. Ependymal cells are identified by markers like CD24, SOX2, NESTIN, FOXJ1 and CD133 as they are also derived from radial glial cells [Shah et al. Cell Function. Cell 173, 1045-1057 (2018)]. These cells generate the choroid plexus and are known as a reservoir of neuro-regeneration. Schwann cells are the primary glia of the PNS. They are similar to oligodendrocytes in function and can be divided as myelinating and non-myelinating Schwann cells on the basis of their functionality. Myelinating Schwann cells produce irregular myelin sheath around neuronal axons in PNS accompanied by some gaps known as nodes of Ranvier. The nerve impulse conduction through saltatory pathway, mediated by oligodendrocytes, is similar to the CNS. Non-myelinating axons have important roles in axon maintenance during myelination and supporting neuron survival and growth. Besides myelination, these cells are also involved in extracellular matrix production, regulating neuromuscular synaptic activity and neuroinflammation by antigen presentation to T-lymphocytes.

Satellite glial cells, also known as amphicytes, cover the neuron cell bodies in the PNS and provide protection, cushioning and cell growth to neurons. These cells regulate the microenvironment of the sympathetic ganglia and support nutrient transport to neurons. Satellite glial cells are similar to astrocytes in terms of electrical properties.

CNS development in all vertebrates is driven by the formation of a neural tube, harbouring the so called embryonic precursors, from which the spinal cord and the brain originate. The neuroepithelial cells of the neural tube divide to expand the neural progenitor pool by symmetric division and act as a cell source for CNS formation. Later, they divide into cells with characteristics, spatial and morphological arrangements similar to astroglia, so called ‘radial glial’ cells (RGCs). RGCs within the ventricular zone (VZ) and the subventricular zone (SVZ) divide asymmetrically to produce two cell populations, one cell is equivalent to the originating radial glia cell (mother cell), and the other is committed to differentiate. This cell is known as the intermediate neural progenitor cell. The intermediate neural progenitors continue dividing in proliferative zones, while post-mitotic neurons migrate to the developing neocortex, thus extending the neurogenesis from the ventricular surface to the pial surface. In the cortex, migrating immature neurons form a time dependent complex layered neocortex consisting of six different layers. Earlier migrating neurons form the deeper layers and later migrating neurons form the superficial layers. RGCs also act as progenitor for glial cells by generating intermediate progenitor cells for astrocyte or oligodendrocyte. This population of progenitor like neurons migrate outward into the overlying white matter and cortex, striatum and hippocampus, where they finally generate astrocytes and oligodendrocytes, thicken the neocortex and support interneuron networks. Unlike neurons, glial progenitors continue to divide during their migration towards target locations. In addition to cell proliferation and migration towards a predetermined brain region, several other events like aggregation of similar cell types, synaptogenesis and neural circuit formation, and selective elimination of cells due to inter-cell competition occurs to stabilize the total number of neurons and glia in human brain.

### Cell-Cell Interactions in Healthy and Diseased Brain

The complexity and neuronal density of the human brain provides an ability to perform intricate neurological functions and respond to the external environment. Current knowledge about the differences in human and other primate brain, such as presence of larger astrocytes with more branches in human brain compared to rodents; provide some clue for exceptional human abilities. Further research recapitulating this complexity and intricate cellular crosstalk in human brain, can provide more insights about how human brain is different from non-primates. It is noteworthy to mention that enhanced memory and cognition has been observed in mice upon transplantation of human astrocytes in murine brain. It indicates that cellular crosstalk between human astrocytes and murine brain cells can support more human-like brain functions in mice. The interactions between neurons and glial cells occurs either by direct contact signalling via ion channels or by long-range signaling through secreted molecules like neurotransmitters, cell adhesion factors and by specialized signaling molecules released from synaptic regions at neurons. This cellular crosstalk has also been observed to regulate organism behavior and cognition. Most of the initial work on cellular crosstalk has been done on neuronal signaling, but the latest advances in neuroscience points to the importance of neuron to glia signaling pathways, alterations to which has been seen to cause neurological disorders. For example, mutations in neuron-astrocyte signaling negatively effects memory and behavior.

A totipotent stem cell, for which zygote act as an example, can generate any cell type including embryonic and extra embryonic tissues. They are located only in early embryonic tissues, originate generally from the first few cell divisions after fertilization and remain present until the eight-cell stage is attained. These cells are the mother cells of the primary germ layers of the early embryo and extra-embryonic structures like the placenta. Besides the production of full embryo, the high proliferative tendency of totipotent and pluripotent stem cells also poses the risk of teratocarcinoma.

Pluripotent stem cells can differentiate to all embryonic cell types but not extra-embryonic tissues. These cells are present in the inner cell mass of a blastocyst formed by the zygote during embryonic development. These cells can be characterized by the expression of proto-oncogenes like C-MYC and stem cell-specific transcription factors such as octamer-binding transcription factor 4 (OCT4), kruppel like factor 4 (KLF4), NANOG and sex determining region Y box 2 (SOX2). The pluripotent cell fate can be captured in vitro in embryonic stem cells (ESCs). ESCs are obtained from the inner cell mass of blastocysts.

Multipotent stem cells are cells that can give rise to a more limited number of specific lineage cells. These are produced from ESCs after primitive streak formation and persist as adult stem cell populations. They are responsible for tissue healing after injury in adult organism. The best example of multipotent stem cells are hematopoietic stem cells.

Unipotent Stem Cells are cells that can differentiate only into one specific lineage of the adult organism and thus have the strictest differentiation potential. Examples of unipotent stem cells are endothelial, corneal or spermatogonial stem cells.

Induced Pluripotent Stem Cells (iPSCs)

Yamanaka et al. identified four genes namely OCT3/4, SOX2, C-MYC and KLF4, that are sufficient to convert a fibroblast into a stem cell.

Genome engineering refers to alterations including DNA insertion, deletion, modification, or replacement in an organism's genetic code. Gene editing techniques consist of transfection of plasmid vectors, transduction with viral-derived vectors, and the more recent site-specific nuclease approaches, including the clustered regularly interspaced short palindromic repeat (CRISPR)—CRISPR associated (Cas) 9 system. These approaches make it possible to generate isogenic and mutant cell lines and study specific cell phenotypes in vitro. Viral vectors transfer transgenes easily and efficiently. However this approach suffers from some limitations like random integration, gene silencing and difficulty in controlling transgene copy numbers. An alternative strategy which researchers have developed is the incorporation of genes of interest at predefined chromosome locations called safe harbor loci. Since alterations at safe harbor locus do not affect crucial cell functions and phenotype of cells, these loci provide a platform for stable transgene expression with minimum harmful effects. Also, the insulator elements present in safe harbor loci inhibit (to some extend) transgene silencing. However, some reports shows that AAVS1 locus is not as safe as it was thought to be initially, as transgene silencing by DNA methylation was detected in both undifferentiated and hepatocyte-committed hESCs. The most commonly used safe harbor locus in human iPSC (hiPSC) is the adeno-associated virus integration site 1 (AAVS1) locus, the natural integration site of AAV type 2 virus located on chromosome 19. Scientists inserted genes of interest in the AAVS1 locus by nucleofecting iPSCs with plasmids containing coding sequence of gene of interest. Therefore, insertion of DNA material in a safe harbor locus like AAVS1 provides an interesting tool to researchers to overexpress certain transcription factors or genes to differentiate different type of cells like neurons and glia [Neyrinck et al. (2021) cited above], to further stem cell-based disease modelling and therapy.

### Disease Modelling and Stem Cell Therapy

iPSC based disease modelling has been made popular due to unparallel advantages they provide. One of the crucial factors is the retention of the genetic makeup in differentiated progeny which forms the basis of the use of iPSC derived cells in disease modelling. Thus, neural models derived from cells derived from a patient can be used to investigate novel drugs, paving the path to clinical trials, and promoting personalized medication approach. These cells can help in comparing the cellular behaviour and their crosstalk between a diseased and a gene corrected isogenic line.

### Human Pluripotent Stem Cells-Derived Neural Cells

Neural stem cells are excellent candidates to understand embryonic neurogenesis, as well as the creation of model systems recapitulating disease pathology and drug screening platforms. The most accessible and acceptable source of neural stem cells are pluripotent stem cells, ESCs and iPSCs. Continuous protocol optimization for obtaining neural progeny from ESCs and iPSCs has resulted in wide range of protocols for differentiating PSC-derived neural stem cells into different neural cell types.

### In Vitro Cortical Neuron Differentiation

Neural stem/progenitor cells can be derived from iPSCs. This can be achieved by formation of embryoid body (EB) structures containing cells from the three germ layers that follow a similar developmental trajectory as seen in vivo. Despite of numerous EB-based cortical neuron differentiation protocols, they present some limitations, including being time-consuming and resulting in high variability between EBs from one batch and between batches. Another way to differentiate stem cells to neural stem cells is based on mouse stromal feeder cell lines. These cell lines such as PA6 and MS5 secrete several factors directing the fate of stem cells towards neurons. However, as understanding of signaling pathways regulating the initial neural commitment process improved over time. Directly conversion of the stem cells into neural precursor cells without the use of EB/organoid formation nor stromal feeder lines can be done using two small molecules (Noggin and SB431542, <900 Da), to inhibit the SMAD signaling pathway and observed the formation of neural rosettes containing PAX6 positive neural stem cells bordered by PAX6 negative neural crest cells. This results in efficient generation of PAX6 positive neural precursor cells in 19 days without using EB or stromal cell line-based protocols. The generation of fully matured cortical neurons using dual SMAD signaling inhibition (BMP and TGF-β/NODAL signaling pathways), suppresses pluripotency and promoting neural cell differentiation. This generates neurons that expressed layer-specific neuronal markers (CUX1-upper layer marker, CTIP2—deep layer marker) and vesicular glutamate transporter 1 (VGLUT1). These cells also showed mature neuron-specific electrophysiology properties. Therefore, this method is suitable to generate mature cortical glutamatergic neurons which can be used to establish in vitro models of co-culture.

### In Vitro Astrocyte Differentiation

Several efforts have been made to generate astrocytes in vitro from iPSCs. Mature astrocytes can generated in three stages, based on the astrocytic in vivo lineage development [Krencik et al. Nat. Biotechnol. 29, 528-534 (2011)]. The first stage consisted of the conversion of iPSCs to neuroepithelial (NE) cells for ten days in absence of growth factors, followed by transition to astroglia by addition of FGF8, RA or SHH for ten more days. At day 15, rosette shaped precursors were isolated and cultured in suspension culture. The last stage comprised of culturing dissociated neural precursor population in the presence of FGF and epidermal growth factor for further maturation. After 180 days, GFAP, S10003 and NFI-A positive immature astrocytes could be seen and could be used for in-vitro experiments. Based on this protocol, many similar experimental designs have been developed. However, problems like long differentiation timelines and variability in astrocyte population, limited their broad dissemination and application. More recently a number of reports identified some key transcription factors like SOX9, NFI-A and/or NFIkB that regulate the neural progenitor to astrocyte switch, which when overexpress produces homogeneous and functional astrocytes in four to seven weeks. Additionally, the present inventors previously demonstrated that SOX9 overexpression in neural progenitors derived over a period of 12 days from PSCs, is sufficient to induce astroglial differentiation in vitro, within ±20 days. Protocols that can generate functional astrocytes in shorter periods can be applicable to produce more efficient astrocyte culture systems as models for neurological conditions and improve the study of glial cell biology.

### In Vitro Oligodendrocyte Differentiation

iPSCs have also been used to generate oligodendrocytes in-vitro. Several protocols have been established focusing on the in vivo developmental trajectory. The oligodendrocyte precursor cells (OPCs) can be generated directly from the signaling molecule-based specification of stem cells that closely recapitulates the in vivo oligodendrocyte development. For instance, Hu et al. and Wang et al. showed x in vitro oligodendrocyte differentiation by first culturing stem cells as EBs to generate PAX6 positive neural precursor cells and then specifying their cell fate towards oligodendrocyte lineage using sonic hedgehog (SHH) and retinoic acid (RA) as signaling cues [Hu et al. Nat. Protoc. 4, 1614-1622 (2009); Wang et al. Cell Stem Cell 12, 252-264 (2013)]. These signaling molecules converted neural precursor cells to glial progenitors which were then supplemented with fibroblast growth factor 2 to prevent their further differentiation to motor neurons. At day 35, the glial progenitors expressed OLIG2 and NKX2.2 with no functional and morphological similarity with in vivo oligodendrocytes. Therefore, the progenitors were further grown for 8 additional weeks in medium supplemented with growth factors such as neurotrophin 3 (NT3), platelet derived growth factor (PDGF), cyclic adenosine monophosphate (cAMP), insulin-like growth factor 1 (IGF-1), biotin and triiodothyronine (T3). This resulted in generation of SOX10 and PDGFRα positive OPCs. This protocol generated stable populations of mitotically competent OPCs, however, low oligodendrocyte yield and long differentiation timeline limited its application. In 2015, Douvaras et al. modified this differentiation protocol by combining the use of small molecules, using SB431542 and LDN193189 to inhibit transforming growth factor β (TGF-β) and bone morphogenetic protein (BMP) signaling respectively, at an initial phase to accelerate the generation of neural precursor cells and consequently, the generation of OPCs in only 55-75 days [Douvaras et al. Stem Cell Reports 3, 250-259 (2014)]. However, oligodendrocyte differentiation remained inefficient, time-consuming, and highly variable until transcription factor-based differentiation was applied. Recently, transcription factor overexpression in combination with lentiviral transduction or incorporation in the safe harbor, AAVS1, have been used to produce oligodendrocytes from stem cells. Transcription factors such as SOX10 and OLIG2 have been described as master gene regulators in oligodendrocyte differentiation and, when overexpressed in iPSCs, to be capable of inducing 04+ and MBP+ oligodendrocytes in 56 days. When combined with the NKX6.2 transcription factor the differentiation time was reduced to 3-4 weeks. Additionally, the present invention demonstrates that SOX10 overexpression in neural progenitors derived over a period of 12 days from PSCs, is sufficient to induce oligodendrocyte differentiation in vitro, allowing effective production of 04+ and MBP+ cells within 22 days.

### In Vitro Model of Neuron-Glia Co-Cultures

As discussed, neuron glia crosstalk plays an important role in neurodevelopment and maintaining brain homeostasis. Mutations in either neurons or glial cells contribute to dysfunction of this cell-cell interaction and contribute to neurological disease and functional deficits. Glial cells play a dual role in both neuroinflammation and degeneration—their reactive phenotype activation can serve as negative factor for one condition while promoting neuroprotection under other circumstances. For example, astrocyte activation can produce neuroprotection and can also exacerbate neuron injury. Functional neuron-glia interactions are, therefore, essential for maintaining resilience against CNS insults and maintenance of efficient neuronal networks. Modelling these interactions in vitro by use of stem cells will provide a platform to scientists to study these interactions and test novel drug compounds. Several efforts have been made in this direction by either coculturing iPSCs or animal origin neurons and glia together or using medium conditioned by one cell type to support growth of the other cell type(s).

### Overview of Current Neuron-Glia Coculture Studies

| | | | | | |
|---|---|---|---|---|---|
| | | | | Days in | Cell ratio |
| Study | Objective | Method | Source | culture | (Neuron:Glia) |

Neuron-astrocyte coculture

| | | | | | | |
|---|---|---|---|---|---|---|
| 1 | Jones et | Effect of astrocyte | Sandwich co-culture | Mice | 5-7 | 1:1 |
| | al. 2018 | derived factors on | method | | weeks | |
| | | neurons | | | | |
| 2 | Aeber- | Effect of astrocytes | Astrocyte culture | Wistar | 3-4 | 1:100, |
| | sold et | on spiking activity | suspension over | Rat | weeks | 1:250, |
| | al. 2018 | and cell viability of | neuron cultures | | | 1:200, |
| | | neurons | | | | 1:10, |
| | | | | | | 1:5, 1:2 |
| 3 | Zhang | Effect of glutamate | Neurons and | Wistar | 2-3 | 1:8 |
| | et al. | toxicity on neurons | astrocyte co-seeding | Rat | weeks | |
| | 2018 | | (on culture strip | | | |
| | | | insert) | | | |
| 4 | Jacob et | Protocol optimization | Neurons on cortical | Mice | 4-5 | 20:9 |
| | al. 2010 | | astrocytes on | | weeks | |
| | | | coverslips | | | |
| 5 | Kidambi | Effect of astrocyte- | Neurons and | Wistar | 2 weeks | 1:1 |
| | et al. | mediumted oxidative | astrocytes | Rat | | |
| | 2008 | stress on neurons | cocultured on | | | |
| | | | polyelectrolyte | | | |
| | | | multilayer films | | | |
| 6 | Krencik | Effect of astrocyte- | iPSC based asteroid | Human | 5 weeks | 1:1 |
| | et al. | neuron interactions | containing | iPSCs | | |
| | 2017 | on neural circuit | astrocytes and | | | |
| | | | neurons | | | |
| 7 | Soscia | Effect of astrocytes | 3D neuron-astrocyte | Human | 6-7 | 3:25 |
| | et al. | on neuron activity | coculture on MEA | iPSCs | weeks | |
| | 2020 | through MEA system | chip | | | |

Neuron-Oligodendrocyte coculture

| | | | | | | |
|---|---|---|---|---|---|---|
| 1 | Swire et | Protocol optimization | Seeding | Rat and | 5-6 | — |
| | al. 2019 | | oligodendrocyte | mouse | weeks | |
| | | | precursors on DRG | | | |
| | | | neurons | | | |
| 2 | Pang et | Protocol optimization | Co-seeding | Rat | 4 weeks | — |
| | al. 2018 | | precursor cells on | spinal | | |
| | | | coverslip | cord | | |
| 3 | Ristola | Studying neuron- | 3D printing-based | Rat | 5-6 | 9:1 |
| | et al. | oligodendrocytes | compartmentalized | | weeks | |
| | 2019 | interactions | neuron- | | | |
| | | | oligodendrocyte | | | |
| | | | coculture | | | |
| 4 | Mazuir | Protocol optimization | Selective | Wistar | 3 weeks | — |
| | et al. | | detachment of glial | Rat | | |
| | 2020 | | cells using shaking | | | |
| | | | and plating | | | |
| | | | oligodendrocytes | | | |
| | | | over neurons | | | |
| 5 | Leon et | Protocol optimization | Myelinating platform | Human | 10 | 1:1 |
| | al. 2020 | | from hPSCs | iPSCs | weeks | |
| | | | (Sox10_AAVS1) | | | |
| 6 | Nada- | Studying neuron- | Human iPSC derived | Human | 9-10 | 0.315:1 |
| | dhur et | oligodendrocytes | neurons and | iPSCs | weeks | |
| | al. 2019 | interactions in | | | | |
| | | Tuberous sclerosis | oligodendrocyte | | | |
| | | complex | precursors | | | |

Neuron-astrocyte-oligodendrocyte cocultures

| | | | | | | |
|---|---|---|---|---|---|---|
| 1 | Enright | Functional and | In vitro brain-on-a | Rat | 5 weeks | 79:16:5 |
| | et al. | transcriptional | chip | | | |
| | 2020 | characterization of | | | | |
| | | complex neuronal | | | | |
| | | co-cultures | | | | |

Jones et al. Methods Mol. Biol. 814, 341-352 (2012)
Aebersold et al. Front. Neurosci. 12, 94 (2018)
Zhang et al. Mol. Med. Rep. 19, 1521-1528 (2019)
Jacobs & Doering (2009) in Protocols for Neural Cell Culture, 269-283
Kidambi et al. Adv Funct Mater. (2008) 18, 294-301
Krencik et al. Stem Cell Reports 9, 1745-1753 (2017)
Soscia et al. Lab Chip 20, 901-911 (2020)
Swire & Ffrench-Constant Methods in Mol. Biol. 1936, 111-128
Pang et al. Methods in Mol. Biol .. 1791, 131-144 (2018)
Ristola et al. J. Micromechanics Microengineering 29, 13 (2019)
Mazuir et al. J. Vis. Exp. 2020, e60912 (2020)
García-León et al. Nat. Protoc. 15, 3716-3744 (2020)
Nadadhur et al. Stem Cell Reports (2019) 12, 42-56
Enright et al. Scientific Reports volume 10, Article number: 11007 (2020)

### Maturation of Neurons and Glia in Co-Culture Systems

The most conclusive approach to study maturation of neurons in these in vitro systems is to determine the functional quality of neurons by electrophysiological studies using patch-clamp or multielectrode array systems (MEA). The patch clamp technique probes a single neuron to determine its intracellular electrical activity but comes at the cost of mechanical damage to neurons by electrodes, and less information regarding neuronal network formation. On the other hand, MEA facilitates neuron activity detection for longer duration, including neuronal network formation, without inflicting any mechanical injury.

MEA systems are electrophysiology cell-imaging platforms wherein functional neurons are cultured on electronic circuitry. These systems capture neuron activities from the entire population grown on them, and provide crucial information linking brain activity with disease pathology and neurodevelopmental events like neuron myelination. MEA systems have been used in this project for coculturing neurons with astrocytes and oligodendrocytes, to determine how the neuronal activity changes in the presence and absence of glial cells. The neuronal activity in turn can provide information on the maturation of neurons in monoculture and coculture systems.

Microelectrode arrays detect the variation in cellular currents because of ongoing ionic processes within and near the cells. It depends on several parameters like amount and sign of current, separation between microelectrodes and recording place. These currents popularly known as action potentials create neuronal spike when transmembrane potential exceeds certain threshold value, due to synaptic flux. The inward sodium ion flow during action potential generates high negative spike in extracellular action potential recording in contrary to the small positive peak generated by potassium ion flux. Also, neuron bursts can be generated on long-duration action potentials that are followed by quiet period of no activity. These bursts can be stimulated because of environmental or phenotypic factors which can be detected and recorded.

Other parameters include population-wide synchronized bursts, which are caused by activation of clusters of either excitatory or inhibitory neurons and this signal synchronization and functional network formation can be regarded as a parameter to characterize neurons maturity. Therefore, high density MEA systems with high signal quality and spatiotemporal resolution enable researchers to track propagation of action potentials and axon transport in neuronal cultures [Bakkum et al. PLoS One 3, e2088 (2008)] as well as determine the effect of glial cells on neuronal activity in complex 2D cocultures [Shih et al. Stem Cell Res. 102386 (2021); Fukushima et al. J. Biomol. Screen. 21, 54-64 (2016)] and 3D systems [Trujillo et al. bioRxiv 358622; Trujillo et al. Cell Stem Cell 25, 558-569.e7 (2019)].

The present invention aims to increase cellular complexity of human iPSC derived in vitro systems by developing a human triculture system containing hiPSC derived neurons, astrocytes, and oligodendrocytes to better recapitulate the in vivo physiology and function of the brain in a scalable format. The present invention provides a human iPSC based triculture system and is characterized for neuron maturation using MEA for neuronal network functionality, maturation, and cellular composition. This application discloses:
(1) differentiation and characterization of hiPSC-derived neurons (cortical and inducible neurons) and glial subtypes (astrocytes and oligodendrocytes) in mono-culture
(2) medium 1 (neural maintenance medium) is sufficient to allow maintenance of all three cell types first in monocultures and then combined tricultures,
(3) the optimalisation of cell ratio, density, seeding strategy and antibody combinations to characterize and quantify neuronal and glial populations in tricultures,
(4) a higher expression of different cortical layer neuronal markers in triculture compared to neuronal monocultures
(5) a significantly enhanced neuronal electrophysiological activity in the triculture system compared with neuron-only monocultures, represented by more spiking activity and neuronal burst activity.

The present invention demonstrates that coculture of iPSC neurons and glial cells allowed faster neuronal maturation with augmented network formation, better synchronization, and overall, more neuronal activity.

To develop the human based triculture system, cortical neurons, iNGN2-iPSC derived neurons, iSOX9-iPSC astrocytes, and iSOX10-iPSC oligodendrocytes were differentiated and characterized. Transcription-factor based fate specification was used for differentiation of inducible neurons, astrocytes, and oligodendrocytes to enable faster and efficient generation. In line with other protocols for cortical neuron differentiation [Espuny-Camacho et al. Cell Rep. 23, 2732-2743 (2018); Muratore et al. PLoS One 9, 105807 (2014)], decreased expression of pluripotency marker OCT4, upregulation of neural precursor markers like NESTIN and TUJ1, as well as TBR2 were observed (even if the latter was not significantly higher than in iPSC) (FIG. 6A).

Presence of TUJ1, MAP2, and CTIP2 proteins confirmed the successful commitment towards the cortical neuron lineage (FIG. 6B-E). Previous studies indicated very fast generation of forebrain excitatory neurons [Zhang et al. Neuron 78, 785-798 (2013)] by forced expression of the NGN2 transcription factor. However, although hastened differentiation was confirmed, more recent studies suggest that the induced neurons are heterogeneous [Lin et al. bioRxiv 2020.11.19.389445 (2020); Carter et al. Front. Genome Ed. 2, 7 (2020)].

An upregulation of neuron progenitor and neuron-specific transcript expression following doxycycline inducible expression of the NGN2 transcription factor levels was observed (FIG. 6F). Neurons generated on day 14 were positive for SOX2, NESTIN, TUJ1, and MAP2 by immunofluorescence staining (FIG. 6G-J). Thus, a fast neuron-specific fate acquisition, with persistence of more immature cells and cells expressing pluripotency genes was confirmed.

For astrocytes, earlier research showed that induction of the NFI-A, NFI-B and SOX9 transcription factors enable astrocyte differentiation from mouse fibroblasts [Caiazzo et al. Stem Cell Reports 4, 25-36 (2015)]. Other studies followed, by overexpressing different combination of these transcription factors in stem cell derived NPCs [Canals et al. Nat. Methods 15, 693-696 (2018); Li et al. 11, 998-1008 (2018); Tchieu et al. Nat. Biotechnol. 37, 267-275 (2019)]. In the present invention, SOX9 transcription factor was overexpressed at AAVS1 locus at the NPC stage to form astrocytes more similar to human primary astrocytes [Neyrinck et al. (2021) cited above]. The transcript levels in iSOX9 astrocytes for S100β and ALDH1L1 were comparable to human primary astrocytes [Neyrinck et al. (2021) cited above], which showed their astrocyte specific identity (FIG. 7A). RNA expression levels of ALDH1L1 and GFAP did not change during the differentiation timeline, even if at the protein level were EAAT1 and S100β positive cells could be identified (FIG. 7B-E). Additionally, decreased OCT4 expression level and increased SOX9 expression (induced by doxycycline, but persisting following removal of doxycycline) indicated astrocyte-specific lineage commitment (FIG. 7A).

For oligodendrocytes, Ehrlich et al. showed hastened oligodendrocyte differentiation by induction of three transcription factors SOX10, OLIG2, NKX6.2 [Ehrlich et al. Proc. Natl. Acad. Sci. USA 114, E2243-E2252 (2017)]. An increase in transcript level of NESTIN, OLIG1, OLIG2, NKX2.2 and MBP was observed which indicated that cells differentiated to OPCs within 24 days of starting of differentiation. Increased SOX10 expression by doxycycline, was also demonstrated at the RNA level (at the end of doxycycline addition) (FIG. 12F). This is further supported by MBP and 04 immunofluorescence and flow cytometry analysis (FIG. 12G-I).

After characterizing all cell types, a master medium that allowed the maintenance of neurons, astrocytes, and oligodendrocytes was selected. Previous studies from the present inventors have used two media for coculturing neurons-astrocytes (master medium 1) [Neyrinck et al. (2021) cited above] and neurons-oligodendrocytes (master medium 2) [Garcia-León et al. Nat. Protoc. 15, 3716-3744 (2020)] that allowed the generation of electrically functional or myelinating platforms, respectively. Other published rodent based neuron-astrocyte-oligodendrocyte triculture systems also used triiodothyronine (T3) in their master medium to support oligodendrocyte growth and function in tricultures. Master medium 1, and master medium 2 supplemented with T3 were used to test the maintenance of the three cell types in monoculture. No significant differences in transcript level of TUJ1, NESTIN and TBR2 for cortical neurons; S100β, SOX9, ALDH1L1, and GFAP for astrocytes; and NESTIN, OLIG1, OLIG2, NKX2.2, SOX10 and MBP for oligodendrocytes were observed for cells grown in either of the two-master medium (FIG. 8A-C). Also, on the protein level, the percentage of TUJ1+ cells in the neuronal progeny; S100β+ cells in astrocytes and MBP+ cells in oligodendrocytes were similar in both M1 and M2 medium (FIG. 8D-L). This indicated that all three cell types survived in both medium with unchanged specific cell marker expression assessed both at the mRNA and protein levels.

Next, co-seeded tricultures were developed and the morphology of all three cell types were compared between monoculture and triculture system (FIG. 9). It was observed that in triculture system—neurons form small clusters, oligodendrocytes accommodated in the centre between neuron bundles and astrocytes surrounded neurite bundles and cell body clusters (FIG. 9A-C). Also, the arrangement of cells in triculture system depends on the cell type ratio, cell density, and culture duration (FIG. 9D-H). Increasing these parameters supported the formation of cell-clusters with time with apparent increased cell-cell interaction. In longer-term cultures (FIG. 9E-H, FIG. 19), oligodendrocytes appeared to extend their branches to align with the neurites, as can be seen by the colocalising MBP and TUJ1 staining (FIG. 15G). This indicates the initiation of myelination of neurons in triculture.

Culture conditions were also optimized to assess the triculture system by testing different antibody combinations to characterize and quantify the three cell types. All three cell types can be correctly characterized: TUJ1 for neurons, S100β for astrocytes and O4 or MBP for oligodendrocytes (FIG. 18). S100β staining clearly was present also in oligodendrocyte nuclei besides the astrocyte cytoplasm (FIG. 10-14). This can be caused by the persistence of immature OPCS in triculture system, which are known to display nuclear expression of S100β. This was addressed by evaluating antibodies against additional specific markers for astrocytes like GFAP. As GFAP expression was seen faint in iSOX9 astrocyte monocultures [Neyrinck et al. (2021) cited above], its expression was not expected in the triculture system. However, other research groups using astrocytes from different sources demonstrated GFAP staining as a hallmark to characterize astrocytes in three-cell cocultures. Therefore, GFAP staining was performed combined with TUJ1 for neurons and MBP for oligodendrocytes in tricultures 2 and 3 weeks after all cells were plated. Specific staining in each respective cell type was observed (FIG. 19). Further, cell culture parameters were optimised, such as cell density, ratio and seeding strategy (FIG. 10-19). Plating the cells in lower densities, e.g., at 2,500 and 5,000 per cell type, resulted in cultures where cells, especially neurons struggled to grow neurites and form networks (FIG. 9A-B, FIG. 10-13B). Higher cell densities, i.e., 30,000 cells per type resulted in clustering of each cell types which was not suitable for image quantification (FIG. 9D, FIG. 18). Therefore, cell ratios with lower cell densities were more homogeneous and quantifiable. Two different astrocyte populations (day 27 and day 37) in two different experiments were used to control the impact of high astrocyte proliferation on survival of neurons and oligodendrocytes in tricultures (FIG. 10-14). As expected, tricultures containing day 27 astrocytes, that are still more proliferative, contained a higher fraction of S100β+ cells than tricultures with day 37 astrocytes.

Previous studies highlighted the strong potential of iNGN2 driven neuronal progeny to serve as models for neuron electrophysiological measurement approaching those of neurons in vivo, and a shorter cell culture duration. [Nehme et al. Cell Rep. 23, 2509-2523 (2018); Cheng et al. Curr. Protoc. Hum. Genet. 2017, 1-21 (2017); Ho, et al. Methods 101, 113-124 (2016); Liu et al. Nat. Commun. 4, (2013)]. Also iNGN2 progeny was used within the human triculture system with astrocytes, and oligodendrocytes using the optimized culture conditions from the cortical neuronal progeny from iPSCs (FIG. 14). With respect to neuron morphology, more clustering and less percentage of iNGN2 neurons was observed than cortical neurons in tricultures. The lower percentage of neurons in tricultures can be due to inefficient neuron quantification in the cell clusters. Similar to cortical neuron tricultures, more oligodendrocytes were found in cocultures with a lower number of astrocytes, possibly due to the fact that more space was available for oligodendrocytes.

The effect of co-culture of neuronal progeny and glial progeny on neuronal maturation was investigated. Previous studies demonstrated that the presence of glial cells can induce faster neuron maturation, in terms of expression of different layer neuronal markers and synchronized neuronal activity. To evaluate whether the presence of both astrocytes and oligodendrocytes positively affects neuronal maturation, the presence of two cortical neuronal markers (CTIP2 and SATB2) was assessed in the triculture compared with cortical neurons monoculture after three weeks (FIG. 15A-D). Herein, the percentage of CTIP2+ and SATB2+ cells in cortical neuron triculture for three weeks increased (FIG. 15E). CTIP2 and SATB2 intensity was also higher in these cells at the end of third week compared to monoculture (FIG. 15F); supporting the importance of glial cells in neuronal maturation.

For iNGN2 neuron triculture, a pilot MEA experiment was designed to investigate neuronal maturation in terms of electrical activity, when cocultured with matured glial cells in triculture compared to iNGN2 monoculture. To evaluate the effect of cell-electrode interface on morphology of neurons and glia, the cell morphology co-seeded and sequentially seeded tricultures as well as neuron monoculture were compared (FIG. 20) and assessed neuronal activity by MEA recordings for 5 weeks. The present invention shows for the first time electrical activity in iNGN2 neurons already after 14-21 days from the iPSC stage only when differentiation was done in the presence of mature astrocytes and oligodendrocytes, while no electrical activity was observed for iNGN2 monocultures for up to 5 weeks after the iPSC stage (the latter consistent with published reports [Neyrinck et al. (2021) cited above].

This example shows: 1. neuron monoculture displays low/no electric activity in terms of neuronal spikes, percentage of active spiking electrodes, neuronal bursts and percentage of active bursting electrodes in comparison with triculture system, 2. higher density cultures are less suitable for long-term MEA recording due to decreasing neuron activity in tricultures and neuron clusters that form 3D structures (more pronounced in monoculture than triculture system), 3. neuron activity is different in sequential seeding and co-seeding tricultures. More neuronal activity occurred in low cell density sequential seeding than co-seeding at weeks 4 and 5 which indicates that seeding astrocytes on neurons-oligodendrocytes coculture induce better neuronal maturation (FIG. 15, 21). One possible explanation for this observation can be a better neuron network formation and better controlled astrocyte number (due to addition of older more matured astrocytes) in sequentially seeded triculture. Similar studies have been done wherein iNGN2 neuron—iSOX9 astrocyte only cocultures were analysed by MEA. Neuronal activity was observed after 40 days of coculture, while the tricultures of the present invention show neuronal activity as early as 2-3 weeks after coculture.

Until now, electrophysiological research has mainly involved assessing the effect of astrocytes on neurons but few if any studies have assessed the role of oligodendrocytes in this process.

The present invention discloses the development of a human iPSC derived triculture model system comprising neurons, astrocytes, and oligodendrocytes. This triculture system has been characterised for maturation of neurons by evaluating the expression of different cortical layer neuron markers and most interestingly, improved electrophysiological activity assessed by MEA. This model can be used for myriads of applications ranging from studying neurodevelopment, neurological disease to drug discovery research in both academia and industry.

This triculture model system can be used to model several neurological disorders in vitro, to study the progression of disease and the interaction of diseased and non-diseased cell types. Current available 3D models like brain organoids and neuron spheroids recapitulate certain crucial aspects of human neurodevelopment, but glial cells develop only late (many weeks/months) in cultures, which in turn are generally accompanied by other problems like lack of vascularization and necrosis. In comparison, complex coculture models like the human triculture presented here has the advantage of coculturing pre-differentiated neuronal and glial progeny at the same time and thus present a high potential in understanding neuron-glia interactions in 3D environment. As the complexity of the 3D triculture would be much higher in a 3D than the 2D triculture system, it will likely also better recapitulate the in vivo environment and provide more reliable information.

To create a 3D triculture system, neurons, astrocytes, and oligodendrocytes can be encapsulated within human matrigel or other hydrogels and further allowed to mature in cultures. It could also be built in a microfluidic device to control the microenvironment and further support efficient cell maturation. Alternatively, 3D tricultures can be grown by hanging drop methods. Such a 3D triculture system can be more extensively characterised using advanced techniques such as spatial transcriptomics and single cell RNA sequencing in comparison with similar data from human developing and mature brain, to understand better cell lineage development. This approach can be further complemented with addition of other important cell types like endothelial cells and microglia to develop more complex cultures, opening broader opportunities in neuroscience research.

The developed MEA-integrated human triculture system can also be used to elucidate neuron-oligodendrocytes and neuron-astrocyte interactions, to study the effect of mutations in any of the glial cells on neuronal function/activity. Additionally, MEA-integrated triculture system can also be extended to pharmacological drug screening to test novel molecules neurological disorders linked with problems in neurons, astrocytes, and/or oligodendrocytes.

### EXAMPLES

### Example 1. Methods and Materials

Human iPSC Culture

All hiPSC lines (SIGi001-A, Sigma) (iSOX9-iPSC generated from by insertion of the TET-ON-SOX9 cassette in the AAVS1 locus of the SIGi001-A line [[Neyrinck et al. (2021) cited above]; iSOX10-iPSC generated by insertion of the TET-ON-SOX10 cassette in the AAVS1 locus of the SIGi001-A line [Garciá-León et al. (2020) cited above]; and BIONi010-C+NGN2 #I7-26, contained a TET-ON-NGN2 cassette in the AAVS1 locus of the BIONi010-C line; Bioneer) were maintained in E8 medium (Gibco) supplemented by Penstrep (Gibco) and E8 supplement (Gibco), at 37 degrees ° C. with 5% CO2 and 5% O2. When iPSCs were 80% confluent, cells were harvested using Ethylenediaminetetraacetic acid (EDTA) (Gibco) and replated on new Matrigel (Life technology) coated plates.

### Monocultures

Cortical neurons, induced neurons, astrocytes, and oligodendrocytes were differentiated from their parent iPSC lines. During each protocol, the cells were placed at 37 degrees Celsius, 5% CO2, 95% humidified incubator and medium replacement was done every other day. The medium composition used for cell differentiation protocols has been summarized in table 1.

### Cortical Neuron Differentiation

Cortical neurons were differentiated from hiPSCs (SIGi001-A, Sigma) following the protocol adapted from Shi et al. Nat. Protoc. 7, 1836-1846 (2012). The timeline for cortical neuron differentiation with brightfield images representing different stages of differentiation, is described in FIG. 1. iPSCs were passaged using accutase (Sigma) and plated at a density of 0.25 million cells/cm2 in mTESR (StemCell) spiked with RevitaCell (Gibco) on matrigel coated plates. For 11 days, neural induction was done by using the combination of small molecules-1 uM LDN193189 (Milteny) and 10 uM SB431542 (Tocris) with neural maintenance medium (NMM) (neural induction medium, table 1). Between day 11 and 15, bFGF2 (PeproTech, 20 ng/ml) was added to the NMM medium to induce neuronal progenitor proliferation. Cultures were purified by three consecutive dispase (Roche) treatments allowing neural rosette formation. On day 27, 5,000-10,000 cells/cm2 neural precursor cells were seeded using accutase, into new matrigel plated cells in NMM supplemented by RevitaCell and maintained until day 100. Also, some cells were cryopreserved in 10% DMSO (Sigma) for future use. Immunostaining was done on day 40 and day 60 neural progenitor cells to characterize for neuron specific markers (TUJ1 and MAP2 (day 40), TUJ1 and CTIP2 (day 60)).

Induced Neuron (iNGN2) Differentiation

The BIONi010-C+NGN2 #I7-26 iPSC line (abbreviated iNGN2) was used to generate neural progenitor population by NGN2 overexpression induced by doxycycline treatment [Neyrinck et al. (2021) cited above]. The timeline for iNGN2 differentiation with brightfield images representing different stages of differentiation, is described in FIG. 2. The cells were expanded and allowed to grow until they were 80-90% confluent. On day −1, they were plated in mTESR medium supplemented with RevitaCell at 100,000 cells/cm² on matrigel coated plates. Cells were treated with doxycycline (3 mg/ml) in NBM medium for 6 days to induce overexpression of the NGN2 transcription factor. On day 4, cells were dissociated using accutase and re-plated at different densities (75,000-300,000 cells/cm2) on matrigel-coated plates. After 6 days of doxycycline addition, wells with neural progeny were cryopreserved in 10% DMSO for future use. The remaining cells were maintained in culture in NMM for 3 weeks and NBM for an additional 3 weeks. iNGN2 progeny on day 14 were replated at 20,000 cells/well in matrigel coated 96 well plates and characterized after a week by immunofluorescence microscopy.

### Astrocyte Differentiation

The iSOX9-iPSC generated by insertion of the TET-ON-SOX9 cassette in the AAVS1 locus of the SIGi001-A line, was used for to generate astrocyte progenitor population by SOX9 overexpression induced by doxycycline treatment [Neyrinck et al. (2021) cited above]. The timeline for astrocyte differentiation with brightfield images representing different stages of differentiation, is described in FIG. 3. iSOX9 iPSC were plated at 100,000 cells/cm² on matrigel coated plates and allowed to grow until they were 80-90% confluent. On day −1, cells were passaged using accutase and 150,000 cells/cm2 cells were replated in mTESR medium supplemented by 1× RevitaCell. Neural induction of 12 days was done by addition of neural induction medium containing 1 uM LDN193189 and 10 uM SB431542. On day 12, the cells were detached using accutase and replated at 65,000 cells/cm2 in matrigel coated plates in NMM supplemented with 1× RevitaCell and 20 ng/ml bFGF. The day afterwards, astrocyte maturation medium supplemented with 3 μg/ml doxycycline was given for 6 days to induce the SOX9 transcription factor. When cells reach 90% confluency, final plating was done at a density of 20,000 cells/cm2 using accutase. Some cells on day 18 were cryopreserved in maturation medium supplemented with 20% DMSO. The remaining cells were maintained in culture in astrocyte maturation medium containing 500 uM N-acetyl-L-cysteine (Sigma), 0.1 mM cAMP (Sigma), 10 ng/ml CNTF (Peprotech), 10 ng/ml BMP4 (Peprotech), 5 ng/ml HB-EGF (Peprotech), for 3 more weeks for astrocyte maturation. Astrocytes were also plated at 5000 cells/well in matrigel coated 96 well plates on day 18 and characterized after a week by immunofluorescence microscopy for EAAT1, ALDH1L1, S100β and GFAP.

### Oligodendrocyte Differentiation

The iSOX10 iPSC cell line, generated from by insertion of the TET-ON-SOX10 cassette in the AAVS1 locus of the SIGi001-A line, was used to generate oligodendrocyte precursor cells (OPCs) by SOX10 overexpression induced by doxycycline treatment. iSOX10 iPSC were plated at 100,000 cells/cm2 on matrigel coated plates and allowed to grow until they were 80-90% confluent. On day −2, iSOX10 iPSC were replated using accutase at a density of 25,000 cells/cm2 in mTESR medium supplemented by 1× RevitaCell in matrigel coated plates. From day 0, neural induction was done for 6 days by addition oligodendrocyte induction medium containing 0.1 μM RA (Sigma), 10 uM SB431542, 1 μM LDN-193189, resulting in neural rosette formation without the need of EB formation. On day 8, day medium containing 0.1 μMRA, 1 μMSAG (Milllipore) was added for 3 days to mimic in vivo oligodendrocyte development. Cells were replated on day 12 on PLO-laminin coated plates using accutase at 50-75,000 cells/cm² in day 8 medium with RevitaCell, and cells treated until day 22 with doxycycline at 3 mg/ml. On day 14, medium was also supplemented with 10 ng/ml PDGFaa (Peprotech), 10 ng/ml IGF1, 5 ng/ml HGF (Peprotech), 10 ng/ml NT3 (Peprotech), 60 ng/ml T3 (Sigma), 100 ng/ml Biotin (Sigma), 1 μMcAMP (Sigma), 2 ug/ml doxycycline (Sigma) to support oligodendrocyte fate transition until day 24. The oligodendrocyte precursor cells (OPCs) on day 24 were cryopreserved in 15% DMSO; and used to identify the percentage of 04 positive cells using FACS analysis. Day 24 OPCs at day 24 were also plated at 5,000 cells/well in PLO-laminin coated 96 well plates and characterized after one week by immunofluorescence microscopy for oligodendrocytes specific markers, 04 and MBP.

### Quantitative Real-Time PCR

RNA was extracted using the RNeasy (Qiagen) kit, and the quality of RNA was analyzed by nanodrop (Isogen Life Sciences). cDNA was made from the RNA samples by reverse-transcription using SuperScript III first strand synthesis system (Life Technologies). Quantitative real-time PCR (RT-qPCR) was performed using the Viia7 Real-Time PCR system (Thermo fisher Scientific) with Platinum SYBR Green qPCR Supermix-UDG (Invitrogen) and the expression values were normalized with the house-keeping endogenous reference gene, glyceraldehyde 3-phosphate dehydrogenase (GAPDH). The PCR cycles consisted of 2 minutes at 95° C., followed by 40 cycles of 15 minutes at 95° C. and 45 minutes at 60° C.

### Flow Cytometry and Cell Sorting

iSOX10 iPSC derived OPCs were washed with DPBS, dissociated into single-cell suspension by accutase and centrifuged at 0.3 RCF for 5 minutes after addition of NMM. The cell pellet was resuspended in 200 ml PBS and divided in two different falcon tubes. Anti-04-APC antibody (Miltenyi; diluted 1/100 in PBS) and IgM-APC antibody (Miltenyi; diluted 1/100 in PBS) was added to the two separate tubes and incubated at 4 degrees for 15 minutes. After washing with PBS, Flow cytometry was performed using the FACSCanto HTS flow cytometer (BD Biosciences) and the obtained data were analyzed using FACSDiva (version 6.1.2) software.

### Immunocytochemistry

Cells plated in 96 well plate (Greiner-Bio) were washed 3 times with PBS (Thermo Fisher) and were fixed using 4% paraformaldehyde (PFA) (Thermo Fisher) for 20 minutes at room temperature (RT). The fixed cells were washed 3 times with PBS with 5 minutes of interval between them. Cells were then permeabilized and blocked using PBS with 0.1% Triton X-100 (Sigma; diluted in PBS) and 5% goat serum (Dako; diluted in PBS), for 1 hour in dark. The primary antibody (summarized in table 2) prepared in PBS with 5% goat serum, was added, and cells incubated at 4 degrees overnight. Cells were washed 3 times with PBS and incubated with secondary antibodies (table 2) prepared in Dako antibody diluent for 1 hour at RT. Nuclear staining was done by incubating cells with Hoechst (1 in 5000 dilution in PBS) for 20 min at RT in the dark. Cells were washed with PBS 2 times with 5 minutes interval and stored in PBS at 4 degrees in the dark until imaging.

Fluorescent images were taken using the Operetta, PerkinElmer, and images were analyzed with Columbus image data storage and analysis software version 2.9 (PerkinElmer). The software identified different cell types on the basis of intensity, morphology and other parameters like texture and roundness. The cells were selected first on the basis of the presence of nuclear staining, followed by characterization of cell parameters. The quantified data was then plotted by using GraphPad Prism software version 5.04.

### Example 2. Cocultures

### Medium Composition

Monocultures of day 38 neural precursor cells (NPCs), day 27 iSOX9-astrocytes and day 24 iSOX10-OPCs were cultured in M1 and M2 medium, respectively (for composition see table 3), in matrigel coated 96 and 6 well-plates for a week with medium changes every other day. On day 8, cells were characterized by RT-qPCR and immunofluorescence microscopy for specific markers. The medium allowing appropriate expression of molecular markers, and cell survival of all three-cell populations in coculture system was defined as master medium for all subsequent tricultures.

### Triculture System

Triculture systems were set up by two different cell seeding strategy: co- and sequential seeding at one and two time points to optimize cell survival, differentiation, and co-maturation of all three cell types together.

### Co-Seeding of all Three-Cell Population:

Day 38 SIGi001-A NPCs, day 27-day 37 iSOX9-astrocytes and day 24 iSOX10-OPCs were seeded together in M1 medium supplemented with RevitaCell (1/100) in PLO-laminin coated 96 well plate at different cell densities and cell ratios (table 3). M1 medium supplemented by with 3 mg/ml doxycycline was added to triculture for 1 week, which was replaced by only M1 medium for the following 2 weeks (protocol in FIG. 5). The co-seeded triculture was allowed to mature for 3 weeks and immunofluorescence imaging with cell specific antibody combinations (table 4) was performed every week.

### Sequential Seeding of Neurons, Astrocytes, and Oligodendrocytes at Two Timepoints

Day 38 SIGi001-A NPCs, day 27/day 37 iSOX9-astrocytes and day 24 iSOX10-OPCs were used for sequentially seeded tricultures using the protocol shown in FIG. 5. Three cell combinations: only iSOX10-OPCs (sequential seeding II), SIGi001-A NPC-iSO10-OPCs— (sequential seeding II.b), iSOX9-astrocytes-iSOX10-OPCs (sequential seeding II.c); were seeded first in PLO-laminin coated 96 well plate at different cell densities and ratios (table 4); followed by addition of the residual cell types: iSOX9-astrocytes, SIGi001-A NPCs, and SIGi001-A NPC-iSOX9-astrocytes after 1 week. The cells were maintained in medium 1 with 3 μg/ml doxycycline for one week. At day 8, the cells were fixed and immunofluorescence imaging with cell specific antibody combinations (table 2) was performed.

Tricultures Containing BIONi010-C iNGN2 NPCs, iSOX9-Astrocytes, and iSOX10-OPCs

The triculture with BIONi010-C iNGN2 neurons was set-up with iSOX9-astrocytes and iSOX10-OPCs with the protocol optimized for the cortical NPC triculture system. Day 14 BIONi010-C iNGN2 NPCs, day 37 iSOX9-astrocytes and day 24 iSOX10-OPCs were seeded as co- and sequential seeding with different cell density and ratio (table 4). M1 medium supplemented with 3 μg doxycycline was used for 1 week in both seeding strategies. Immunofluorescence was performed after 8 days with suitable antibody combinations (table 2).

### Maturation of Triculture Systems

Maturation of SIGi001-A NPCs in the triculture system was evaluated by identifying expression of the cortical neuron markers, CTIP2 (Layer V), and SATB2 (Layer IV), using immunofluorescence microscopy. For characterizing the BIONi010-C iNGN2 neuron maturation, tricultures were set up on MEA plates and neuronal activity in coculture and monoculture conditions evaluated.

### Deep Layer Neuron Markers in Cortical Triculture System

Tricultures containing SIGi001-A NPCs, day 27 iSOX9-astrocytes, and day 24 iSOX9-OPCs day 24 with different cell ratios and densities (table 4) were cultured in a PLO-laminin 96 well plate for 3 weeks. In addition, SIGi001-A NPC monocultures were used to compare maturation in tricultures. Immunofluorescence imaging was performed every week with antibodies against CTIP2 and SATB2 for neurons, S100β for astrocytes and MBP for oligodendrocytes.

Multielectrode Array Analysis of the iNGN2 Triculture System

Day 4 BIONi010-C iNGN2 neuron precursor cells, day 46 iSOX9-astrocytes and day 24 iSOX10-OPCs were plated in 24 well MEA plate containing 12 microelectrodes per well (Multichannel systems). Cells were plated at three different densities and one cell ratio (table 4), using co-seeding and sequential seeding approaches. For co-seeding the three cell types, cells were seeded on the same day while in sequential seeding, iNGN2 progeny and iSOX10-OPCs were plated first on day −1 followed by addition of iSOX9-astrocytes after a week. Medium was changed every other day with master medium 1 supplemented with 3 mg/ml doxycycline for first week, followed by only master medium 1 for rest 4 weeks. MEA recording was done once a week on the Multi-well System MEA Interface board multiboot recorder (Multichannel System). The electrophysiological data was analysed on Multiwell-analyser software version 1.8.7 (Multichannel system).

### Statistical Analysis

One-Way ANOVA, multiple comparisons was used to compare the mean of different groups for immunofluorescence intensities and RT-qPCR data. Three technical replicates and two biological replicates were taken for analyzing gene expression during mono-culture differentiation of SIGi001-A NPCs, iNGN2 NPCs, iSOX9-astrocytes, and iSO10-OPCs (FIGS. 5-9). Statistical differences were considered significant at a p-value<0.05, performed with Bonferroni correction for multiple comparison in GraphPad Prism version 5.04 software for Windows (GraphPad Software, San Diego California USA). Data is shown as the mean±standard deviation and significant differences are marked *, **, *** for p values<0.05, <0.01, or <0.001, respectively. For immunostainings of monocultures and tricultures, three technical replicates and two biological replicates with multiple fields covering at least 70 percent of the well were considered for analysis. The analysis of immunofluorescent microscopy images was performed using Columbus image data storage and analysis software. Only one MEA-based experiment was performed with 3 separate technical replicates for each coculture conditions and 2 technical replicates for neuron monoculture. MEA analysis was done on Multiwell system analyser and data is shown as medium for each condition.

### Example 3. Characterization of iPSC Derived Neurons, Astrocytes, and Oligodendrocytes

Neurons (from wild-type iPSC and iNGN2 iPSC), astrocytes (iSOX9 iPSC) and oligodendrocytes (iSOX10 iPSC) were differentiated by growth factor-based and transcription factor-based differentiation protocols from their respective iPSC cell line. To characterize the differentiated cells, RNA expression levels of pluripotency (OCT4), neural progenitor (NESTIN) and specific markers for each cell type, were evaluated at different timepoints during differentiations via RT-qPCR. Ct values were compared to the expression value of a housekeeping gene GAPDH, and ΔCt values was plotted as shown in FIGS. 6A, 6F; 7A and 7F.

### Cortical Neurons

Cortical neurons were differentiated from SIGi001-A wildtype hiPSCs using a protocol adapted from Shi et al. [Shi, Y., Kirwan, P. & Livesey, F. J. Directed differentiation of human pluripotent stem cells to cerebral cortex neurons and neural networks. Nat. Protoc. 7, 1836-1846 (2012)]. Expression of the pluripotency marker OCT4 was downregulated along the differentiation timeline (day 100: ΔCt=−5.341±0.9567) compared to day −2 (ΔCt=1.259±1.158) (p<0.05). Transcript levels of NESTIN were upregulated around two times on day 20 (ΔCt=−2.010±0.8491) compared to day −2 (ΔCt=−3.851±0.5735), and gradually decreased until day 100 (ΔCt=−4.280±0.8252). TUJ1 expression increased significantly along the course of differentiation timeline until day 80 (ΔCt=−2.153±0.7884), reaching three times the value compared to day −2 (ΔCt=−6.166±0.3954) (p<0.05). Transcript levels of another neuronal marker, TBR2, varied along the differentiation with highest expression on day 60 (ΔCt=−15.75±0.3055) compared to day −2 (ΔCt=−21.37±1.797) (FIG. 6A).

Neurons were spread all across the microscopic fields and displayed extensive arborization. Cells stained positive for both the TUJ1 and MAP2 protein (FIG. 6B-C) markers. Small clusters of neurons were also observed along with individual cells.

Expression of TUJ1 was observed in both neuronal cell bodies and neurites, whereas MAP2 was only observed in dendrites. Also, neurons on day 60 were stained for TUJ1, and CTIP2 to evaluate their maturation in monocultures (FIG. 6D-E). this demonstrated presence of thick axon bundles connecting cell clusters, and nuclear expression of deep layer neuron marker, CTIP2.

NGN2 Induced Neurons (iNGN2)

BIONi010-C iNGN2 iPSCs were differentiated towards neurons following the protocol described in FIG. 2 [Neyrinck et al. (2021) cited above]. Expression of OCT4 was downregulated approximately 10-fold on day 7 (ΔCt=−10.67±2.570) and week 6 (ΔCt=−10.47±5.302) compared to day −1 (ΔCt=−1.849±0.6230). NESTIN expression increased two times on day 7 (ΔCt=−2.764±0.9504) and then decreased until the end of differentiation (ΔCt=−3.150±2.167) which indicates maturation of neural progenitor cells by week 6. TUJ1 expression increased approximately six times on day 7 (ΔCt=−1.334±0.1761) and week 6 (ΔCt=−2.826±0.7163) compared to day −1 (ΔCt=−6.560±0.1633), demonstrating neuronal fate specification. Expression of NGN2 was increased 66% from day −1 (ΔCt=−18.50±0.01273) on day 7 (ΔCt=−12.10±0.2751) and remained unchanged (ΔCt=−11.73±0.2135) until week 6 (FIG. 6F).

Presence of cells staining positive for SOX2, NESTIN, TUJ1 and MAP2 was observed using immunofluorescence microscopy (FIG. 6G-J). iNGN2 progeny on day 21 were positive for all four markers. The cells were arranged as neuronal rosettes, with small cell clusters distributed across the entire cell culture indicating presence of immature neurons on day 21. Expression of TUJ1 and MAP2 was observed in all neurites (FIG. 6G-H). NESTIN and SOX2 expression were observed in the cell body and in the nuclei of neural day 21 iNGN2 progeny respectively (FIG. 6I-J).

Astrocytes iSOX9 iPSCs were differentiated to astrocytes using the protocol depicted in FIG. 3 [Neyrinck et al. (2021) cited above]. Expression of OCT4 was 6.5 times lower on day 18 (ΔCt=−8.941±0.9157); and 11 times lower on day 27 (ΔCt=−10.00±0.3005) compared to day −1 (ΔCt=−1.392±0.3486). NESTIN expression doubled on day 18 (ΔCt=−2.011±0.1322) compared to day −1 (ΔCt=−4.242±0.4059) and then decreased on day 27 (ΔCt=−3.039±0.6314) indicating maturation of NPCs to astrocytes. SOX9 expression was four times higher on day 18 (ΔCt=−2.907±4.309) after doxycycline treatment, compared to day −1 (ΔCt=−8.870±0.3125), demonstrating doxycycline induced upregulation of driving transcription factor; and remained high on day 27 (ΔCt=−7.059±0.5226) demonstrating endogenous SOX9 expression in astrocytes. A significant increase of around four times in S100β expression was observed on day 18 (ΔCt=−4.816±1.039) and around 60% on day 27 (ΔCt=−8.005±0.7121), compared to day −1 (ΔCt=−13.08±0.7913) demonstrating glial cell directed fate specification (p<0.01). No significant differences were detected for GFAP and ALDH1L1 expression along the differentiation timeline (FIG. 7A).

Astrocytes presented elongated morphology and positive expression of S100β, EAAT1, ALDH1L1 and GFAP (FIG. 7B-E)). The expression of S10003 was observed both in the cytoplasm (high) and nuclei (less extent) (FIG. 7B), whereas EAAT1, ALDH1L1 and GFAP expression was exclusively in the cytoplasm (FIG. 7C-E).

### Oligodendrocytes

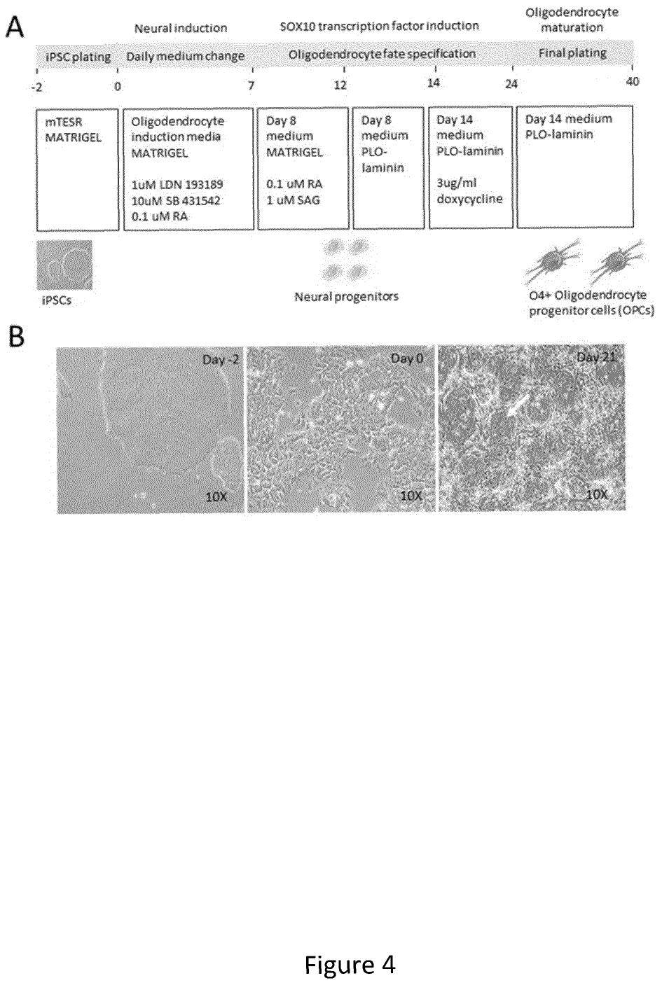

SIGi001-A iSOX10-iPSCs were used to generate oligodendrocytes using the protocol shown in FIG. 4 [Garcia-León et al. (2020) cited above]. Transcript levels of OCT4 remained unchanged until day 12 (ΔCt=−3.518±0.5657 on day 12 versus ΔCt=−3.837±0.1810 on day −2); however, on day 24 (ΔCt=−4.228±1.829) it presented a 10% decrease. NESTIN expression doubled from day −2 (ΔCt=−4.265±0.2659) to day 12 (ΔCt=−2.896±1.202) and remained stable until day 24 (ΔCt=−2.019±0.1047). The expression level of MBP remained low and unchanged until day 18 (ΔCt=−15.36±1.830) but was induced 8 times on day 24 (ΔCt=−2.440±1.275) compared to day −2 (ΔCt=−15.11±0.4780) (p<0.01). OLIG1 expression also increased 80% on day 18 (ΔCt=−12.14±2.567) and 2 times on day 24 (ΔCt=−7.483±0.7022) compared to day −2 (ΔCt=−15.18±0.2263) (p<0.05). In addition, expression of OLIG2 increased 30% on day 18 (ΔCt=−10.77±2.945) from day −2 (ΔCt=−15.06±1.388) and was found to be doubled on day 24 (ΔCt=−5.960±1.498) indicating the presence of oligodendrocyte progenitors. NKX2.2 expression was also increased by 20% on day 18 (ΔCt=−12.74±5.827) and 3 times on day 24 (ΔCt=−5.960±1.498). Finally, SOX10 transcript levels increased from day −2 (ΔCt=−8.040±0.3910) to day 18 (ΔCt=−6.545±1.942) and was 8-fold higher on day 24 (0.6250±0.4992) than day −2, demonstrating doxycycline induced overexpression (p<0.05) (FIG. 7F).

Oligodendrocytes presented a typical round morphology with an intricate and complex pattern. Cells stained positive for both 04 and MBP (FIG. 7G-H). MBP and 04 staining were observed in the cytoplasm of oligodendrocytes. Additionally, flow cytometry using a conjugated anti-04-APC antibody showed that on day 24, 96.9% of oligodendrocytes were 04 positive compared to 1% 04 expression in unstained negative control oligodendrocytes (FIG. 7I).

The present example shows that neurons and glial cells derived from their parental iPSC lines presented the expected morphology as well as gene expression pattern.

### Example 4. Both M1 and M2 Medium Supports Survival of all Three Cell Types in the Triculture System

Cell culture medium composition plays a crucial role in cell biology, gene expression, and survival. To create tri-cultures mimicking brain, it has to recapitulate the fundamental principles of brain physiology and allow cells to mature and become functional in a time-dependent fashion. RNA levels of specific cell markers in NPC progeny, astrocytes and oligodendrocytes were evaluated following 7 days of culture in M1 and M2 medium via RT-qPCR. The ΔCt values were calculated with respect to GAPDH as house-keeping genes.

Transcript levels of TUJ1, NESTIN and TBR2 did not show significant differences for NPCs maintained in either M1 and M2 medium (FIG. 13A). For oligodendrocytes, expression of SOX10, MBP, OLIG1, OLIG2 and NKX2.2 were also not significantly different between M1 and M2 medium (FIG. 13B). Similarly, expression of the astrocytic marker genes S100β, SOX9, ALDH1L1 and GFAP were not significantly different between M1 and M2 medium when iSOX9-astrocytes were cultured for 7 days (FIG. 13C).

Transcript levels of TUJ1 (ΔCt=−4.980±0.3185 (M1); ΔCt=−4.530±0.1155 (M2)), NESTIN (ΔCt=−4.550±0.1310 (M1); ΔCt=−4.914±0.001500 (M2)) and TBR2 (ΔCt=−17.38±1.313 (M1); ΔCt=−15.92±0.2290 (M2)) did not show significant differences between M1 and M2 medium for NPCs (FIG. 13A).

For oligodendrocytes, expression of SOX10 (ΔCt=0.1450±0.06930 (M1); ΔCt=0.3100±0.1853 (M2)), MBP (ΔCt=−1.992±1.684 (M1); ΔCt=−2.522±1.025 (M2)), OLIG1 (ΔCt=−8.227±1.276 (M1), ΔCt=−8.319±0.6569 (M2)), OLIG2 (ΔCt=−7.046±2.542 (M1); ΔCt=−7.302±1.952 (M2)) and NKX2.2 (ΔCt=−4.748±0.07425 (M1); ΔCt=−4.634±0.5310 (M2)) were also not significantly different between M1 and M2 medium (FIG. 13B).

Similarly, expression of astrocytic markers S100β (ΔCt=−7.582±0.06750 (M1); ΔCt=−8.543±0.08103 (M2)), SOX9 (ΔCt=−6.763±0.04400 (M1); ΔCt=−6.881±0.04989 (M2)), ALDH1L1 (ΔCt=−19.25±0.1035 (M1); ΔCt=−19.00±0.3008 (M2)) and GFAP (ΔCt=−15.51±0.1360 (M1); ΔCt=−16.15±0.1025 (M2)) were not significantly different between M1 and M2 medium for day 34 astrocytes (FIG. 13C).

Immunostaining demonstrated also no significant differences in the percentage of TUJ1 positive cells in neuron monoculture in M1 (66.81±1.955 TUJ1±% cells) and M2 medium (63.15±6.760 TUJ1±% cells) (FIG. 13D-F). In oligodendrocyte monoculture, the percentage of MBP positive cells was also similar in both M1 (51.63±2.620 MBP±% cells) and M2 (53.12±1.815 MBP±% cells) medium (FIG. 13G-I). Similarly, the percentage of S100β positive cells was not significantly different in M1 (65.52±3.910 S100β±% cells) and M2 (67.34±2.068 S100β±% cells) medium (FIG. 13J-L).

This example shows that M1 and M2 medium are suitable for the maintenance of all three cell types with high survival rate and similar transcript and protein expression.

### Example 5. Human Triculture System Containing iPSC-Derived Cortical Neurons and Glia

The approach presented here combines iPSC technology in addition to genome engineering to create a three-cell co-culture/triculture as a tool to explore human brain development and disease.

The human triculture system was developed using M1 medium. Co- and sequential cell seeding with multiple timepoints, different cell densities and ratios were performed, and different antibody combinations were tested to correctly characterize the cell types and cell population ratio in cocultures.

### Example 6. Neural Cell Morphology Varies in the Triculture System Compared to Monocultures

Cells were observed with morphological features similar to all three cell types in the triculture systems using both co- and sequential seeding of SIGi001-A NPCs, iSOX9-astrocytes, and iSOX10-OPCs. Cells with morphology similar to NPCs were present in the form of clusters all across the microscopic fields (FIG. 9A-D) with dispersed between the neuronal clusters astrocytes and oligodendrocytes, compared to the scattered arrangement of neurons all over the well with no space between cell clusters, in monoculture system (FIG. 13D-E). Neuronal neurites were observed in close proximity to both astrocytes and oligodendrocytes in the triculture system. The morphology of astrocytes was seen to be highly variable in both monoculture and triculture system. Some cells were bifurcated and stellate-like with circular soma, while other were elongated and rod-shaped soma. However, they were found near neuronal neurite bundles in triculture (FIG. 9A-D) compared to their segregated arrangement in monoculture system (FIG. 13J-K). Overall, astrocytes were observed as elongated flat cells covering the bottom of the plate entangled with neurons. On the contrary, oligodendrocytes were present in groups surrounded by neuron and astrocytes clusters in tricultures (FIG. 9A-D) compared to segregated cells seen in monoculture (FIG. 13G-H). The oligodendrocytes were circular in shape, in close contact with neuronal neurites and astrocytes in the triculture system (FIG. 9A-D). Further, the triculture system appeared to have multiple layers of astrocytes and oligodendrocytes compared to planar monolayers in their monocultures systems. Neurons were present in multiple layers in both monocultures and tricultures.

Cell morphology in triculture was also found to be dependent on the cell ratio and density. Within higher density cell cultures (FIG. 9C-D), oligodendrocytes accumulated within neuron-astrocyte clusters possibly due to limited space left, as also observed in later immunofluorescence images (FIG. 10-14). On the contrary, neurons were present as separate cells with shorter neurites at lower cell densities (FIG. 9A-B). Therefore, cell density and ratio acted affect cell morphology in triculture system.

Another factor affecting morphology of cells was culture duration of the triculture system. The neurons tended to form bigger clusters in longer duration tricultures (FIG. 9E-H) with thicker bundles of neurites joining the clusters, similar to long term monocultures (FIG. 6D-E). The big-elongated astrocytes settled near neuron clusters and got intermixed with neurites to form dense bundles (FIG. 4E-H). The oligodendrocytes were seen in the center of neuron-astrocytes clusters, with bigger projections next to neurites as the triculture matured (FIG. 9E). Cell death also increased in the center of big clusters in relation to the duration of triculture system (FIG. 9H).

The example shows that SIGi001-A NPCs, SIGi001-A iSoX9 astrocytes, and SIGi001-A iSOX10 OPCs survived in M1 medium as a triculture system.

The example shows that the morphology of neurons and glial subtypes differs in the triculture and monoculture system and was dependent on cell density and ratio and the duration of culture.

### Example 7. Antibody Combinations to Characterize and Quantify SIGi001-A NPCs, iSoX9 Astrocytes, and iSOX10 OPCs in the Triculture System

SIGi001-A NPCs, iSoX9 astrocytes, and iSOX10 OPCs seeded combined (co-seeding) or at two time points (sequential seeding) were cultured together for a week followed by characterization of the triculture system by immunofluorescence microscopy for specific cell markers: TUJ1, MAP2, TAU, SATB2 and CTIP2 for neurons; EAAT1, ALDH1L1, and S100β for astrocytes; and 04 and MBP for oligodendrocytes (FIG. 18). Different antibody combinations (table 2) were tested on triculture system. It was observed that TUJ1 for neurons, S100β for astrocytes and 04 and MBP for oligodendrocytes were suitable markers for their visualization in triculture system. The combinations, TUJ1+S100β+MBP and TUJ1+S100β+04 showed clear expression of TUJ1 in the neurites and cell body of the neurons; S100β stained the cytoplasm and nucleus of astrocytes; and cytoplasmic expression of 04 and MBP in cytoplasm of oligodendrocytes was observed (FIG. 23). TUJ1+S100β+MBP antibody combination was selected to quantify the percentage of each cell population in tricultures as it supported image quantification in automated screening platform. S100β staining also occurred in nuclei and branches of oligodendrocytes, making quantification difficult. Therefore, an antibody against another astrocyte marker, GFAP was used together with TUJ1 for neurons and MBP for oligodendrocytes, to label astrocytes in long-term duration cultures. This demonstrated clear expression only in the cytoplasm of astrocytes but not oligodendrocytes and therefore, solved the antibody overlap problem (FIG. 19A-D).

The example shows that a tri-culture of SIGi001-A NPCs, iSoX9 astrocytes, and iSOX10 OPCs can be characterized based on specific marker expression using immunofluorescence microscopy.

### Example 8 Optimizing Cell Seeding Strategy, Cell Density and Ratios in Human Triculture System

Tricultures containing day 38 SIGi001-A NPCs, day 27 or day 37 iSOX9 astrocytes, and day 24 iSOX10 OPCs were set up by both co-seeding and sequential seeding at different cell ratios and cell densities (table 4). Astrocytes represented more than 60% of all cells in both co- and sequentially seeded tricultures (FIG. 10-14).

When cells were co-seeded at equal ratios, the maximum number of neurons was observed in culture with the highest overall cell density (10,000 cells per type: 30.41±0.0% TUJ1+ cells, FIG. 10A). The highest number of oligodendrocytes was observed in cultures with the lowest overall cell number (2500 cells/type: 5.432±2.646% MBP+ cells, followed by 5000 cells/type: 2.549±1.608% MBP+ cells, FIG. 10B). More neurons were observed when the ratio of NPC/Astrocyte/OPC was unequal and similar to the in vivo human brain neuron/glia ratio (1:0.5:0.5) (19.64±0.0% TUJ+ cells, FIG. 10C) followed by 1:1:0.5 (16.05±10.21% TUJ1+ cells, FIG. 10D) and 1:0.5:0.25 (13.36±9.238% TUJ1±cells, FIG. 10E); whereas most oligodendrocytes were found in cultures wherein the ratio of astrocytes was the lowest: 1:0.5:0.25 (5.737±0.502% MBP+ cells, FIG. 10E). A more homogeneous cell distribution was observed when astrocytes were plated in at lower numbers (1:0.5:0.25 FIG. 10E) compared to conditions wherein the ratio of astrocytes was higher (1:1:1) (FIG. 10A).

For sequential seeding II.a, where iSOX10 OPCs was plated first, followed by SIGi001-A NPCs and iSOX9 astrocytes addition; the number of oligodendrocytes was high in higher cell density cultures (20.83±0.0% MBP+ cells in 10,000 cells/type, FIG. 11A and 21.8±10.02% MBP+ cells in 5,000 cells/type, FIG. 11B), compared to lower cell density cultures (13.37±0.0% MBP+ cells in 2500 cells/type). For all three cell densities with equal ratio of cells, the TUJ1+ neuron population comprised less than 4% of total cells, indicating that this culture condition does not support NPC/neuron survival or proliferation (FIG. 11A-B).

For sequential seeding II.b, where iSOX10 OPCs and SIGi001-A NPCs were plated first, followed by iSOX9 astrocytes addition. The highest neuron and oligodendrocyte number was observed in cultures initiated with overall higher cell numbers (6.408±0.0% TUJ1+ cells, 15.77±0.0% MBP+ cells in 10,000 cells/type FIG. 12A) compared to lower cell densities (5.551±4.486% TUJ1+ cells, 14.31±9.515% MBP+ cells in 5,000 cells/type, FIG. 12B), when cells were plated at equal ratios. When plated at different ratios, a higher neuron number was observed with higher cell density (9.472±2.852% TUJ1+ cells, 1:1:0.5 FIG. 12C) while a higher oligodendrocyte number was seen in cultures with lower astrocyte numbers (29.92±0.0% MBP+ cells in 1:0.5:0.5, FIG. 12D and 26.21±0.0 in 1:0.5:0.25, FIG. 12E).

For sequential seeding II.c, where iSOX10 OPCs and iSOX9 astrocytes were plated first, followed by SIGi001-A NPCs addition. The highest number of neurons and oligodendrocytes were present in cultures with higher cell densities (6.867±0.0% TUJ1+ cells and 10.41±0.0% MBP+ cells, FIG. 13A) compared to lower cell densities (4.926±6.608%+TUJ1+ cells and 7.024±7.606% MBP+ cells, FIG. 13B) when cells were plated at equal ratios. Following the same pattern as sequential seeding II.a and II.b, the highest number of oligodendrocytes was detected with when low numbers of astrocyte were plated, 1:0.25:0.5 (22.49±0.0% MBP+ cells FIG. 13C), while the highest neuron number was observed at a cell ratio of 1:0.5:0.5 (5.095±0.0% TUJ1+ cells, FIG. 13D)

Overall, more neurons were observed in co-seeded cultures with higher cell density with equal cell ratio and higher oligodendrocyte numbers in culture with lower numbers of astrocytes. Also, sequential seeding II.b and II.c with cell densities having lower numbers of astrocytes, more oligodendrocytes and neurons were observed.

The example demonstrates that co-seeded tricultures contain relatively more neurons and sequentially seeding II.b tricultures contains more oligodendrocytes. The example demonstrates that a human triculture system containing neurons, astrocytes and oligodendrocytes has been developed with optimized cell ratios, density and seeding strategy.

### Example 9. Human Triculture System Containing BIONi010-C iNGN2 Neurons

BIONi010-C day 14 iNGN2 neurons day 14, day 27 iSOX9 astrocytes and day 24 iSOX10 OPCs was used to set up triculture system at different cell ratios and densities (table 4). For co-seeding, the highest number of neurons and oligodendrocytes were observed at a ratio of 1:0.5:0.25 (10.964±0.0% TUJ1+ cells and 1.865±0.0% MBP+ cells, FIG. 14A). For the ratio 1:1:0.5, higher number of neurons were observed in sequential II.b seeding protocol (16.936±0.0% TUJ1+ cells, FIG. 14B) and higher numbers of oligodendrocytes were seen with co-seeding (1.865±0.0% MBP+ cells, FIG. 14C). Importantly, the percentage of TUJ1+ cells and MBP+ cells was lower in tricultures containing iNGN2 neurons than tricultures containing cortical neurons. In summary a human triculture system with induced neurons, astrocytes and oligodendrocytes has been set-up using optimized culture conditions.

### Example 10. Mature and Functional Neurons are Present in Human Triculture System

To evaluate the maturation of neurons in the triculture system, expression of different cortical layer neuronal markers was assessed by immunostaining, and functional neuron activity was characterized in triculture and neuron monocultures by MEA analysis.

CTIP2 and SATB2 positive cells increase more rapidly in cortical neuron tricultures, compared to the cortical neuron monoculture.

Day 38 SIGi001-A NPCs, day 37 iSoX9 astrocytes, and day 24 iSOX10 OPCs were co-seeded in ratio 1:1:1 (10,000 cells per type), maintained for three weeks and immunofluorescence imaging performed for CTIP2 and SATB2 as different cortical layer neuron markers, S100β for astrocytes and MBP for oligodendrocytes. The expression of SATB2 (FIG. 15A-B) and CTIP2 (FIG. 15C-D) increased over time in the triculture system. After one week, 0.8205±0.1553% CTIP2+ cells and 0.6893±0.3604% SATB2+ cells were observed, which increased to 0.9680±0.04895% CTIP2+ cells and 3.986±1.054% SATB2+ cells after two weeks (FIG. 15E). The percentage of CTIP2+ and SATB2+ cells increased further to 19.94±3.209% and 5.094±1.596% after three week, indicating maturation of neurons in the triculture system (FIG. 15E). The average intensity of CTIP2+ cell was lower in monoculture (0.222±0.00) compared to triculture (0.6067±0.05547) indicating higher CTIP2 expression in triculture than monoculture (FIG. 15F). Similarly, the average intensity of SATB2 per cell was lower in monoculture (0.438796±0.00) than triculture (2.519±0.5521), which suggests improved neuronal maturation when NPCs are cocultures with astrocytes and oligodendrocytes (FIG. 15F).

The colocalization of TUJ1 and MBP in the intersecting regions of neuron axons and oligodendrocyte arms was observed which indicates the starting of myelin wrapping (FIG. 15G).

This example shows that the number of CTIP2+ and SATB2+ cells increased in the cortical neuron triculture system with time.

This example shows that The intensity of CTIP2+ and SATB2+ cell staining is higher in cortical neuron triculture, compared to monoculture after three weeks.

### Example 11. BIONi010-C iNGN2 Neurons are Electrically Active in the Triculture System, not in Monoculture System

For tricultures containing BIONi010-C iNGN2 cells, the MEA system was used to detect neuronal electrophysiology either in monoculture and coculture with astrocytes and oligodendrocytes. Day 4 iNGN2 progeny alone and in combination with day 46 iSOX9 astrocytes, and day 24 iSOX10 OPCs were seeded on MEA plates and difference in cell morphology between monoculture and triculture was evaluated (FIG. 20).

After week 1, the cell morphology and arrangement in the triculture was similar to the one observed for the cortical triculture (FIG. 10-13). For example, neuron and astrocytes arranged in bundles with oligodendrocytes in the center. Similar morphology was observed in neuron-oligodendrocyte coculture with smaller neuron bundles possibly due to the absence astrocytes. On the contrary, neuron monoculture had scattered neuron cells with bundles of neuron cell bodies scattered all over the well (FIG. 20A). After two weeks, thick neurite bundles surrounded with oligodendrocytes were seen in both co- and sequential seeded II.b tricultures (FIG. 20B). The neurons started to organize themselves together to form big clusters observed clearly on week 3. By contrast, in sequentially seeded tricultures, cells covered the whole well while co-seeded triculture started forming multilayers. During the fourth and fifth week, no differences in terms of cell arrangement in co-seeded and sequentially tricultures were observed (FIG. 20C-D).

The electrical activity of the tricultures was compared with neuronal monocultures at the same cell density every week for 5 weeks. In general, an increase in both spiking rate and percentage of active spiking channels was observed in both co-seeded and sequentially seeded tricultures for five weeks, while minimal/no spiking was observed in neuron monocultures (FIG. 16, 17C-D).

After 1 week, spike activity was detected in co-seeded triculture (0.21 Hz) in comparison to no activity in sequentially seeded triculture and neuron monoculture (FIG. 16A-D). The spike activity increased in sequentially seeded tricultures after week 2 (0.2375 Hz) compared to co-seeded tricultures (0.1485 Hz) and neuron monocultures (0.063 Hz). After 3 and 4 weeks, co-seeded tricultures had more spike activity (1.542 Hz-week 3 and 1.443-week 4) compared to sequentially seeded tricultures (1.328 Hz-week 3 and 1.185 Hz-week 4) and neuron monocultures (0.32 Hz—week 3 and 0 Hz—week 4). Also, the spike activity depended on cell density of triculture and monoculture systems. For instance, after 4 weeks the spike activity was higher in sequentially seeded tricultures (0.3711 Hz, 75% active spiking electrodes) than co-seeded tricultures (0.2327 Hz, 41.67% active spiking electrodes) and monocultures (0.37 Hz, 4.167% active spiking electrodes) with 50,000 neurons and glia; compared to higher spiking activity in co-seeded tricultures (0.58 Hz, 58.33% active spiking electrodes) than sequential tricultures (0.1947 Hz, 58.33% active spiking electrodes) and monocultures (0.37 Hz, 4.167% active spiking electrodes) with 75,000 neurons and glia (FIG. 16A-D).

No burst activity was observed in neuron monocultures (FIG. 17A-D). The highest burst spike rate was observed in week 4 sequential cocultures at low cell density (39.4 bursting activity and 41.67% active bursting channels). This was higher than in low density co-seeded tricultures (27.58 bursting activity and 25% active bursting channels). For sequentially seeded tricultures containing 30,000 neurons and glia, bursting continued to increase until week 5 (FIG. 17C-D). Some network bursts were also observed during week 4 and 5 in sequential-seeded tricultures that indicated the initial point of synchrony (data not shown).

The example shows that increased neuronal activity (both neuronal spikes and bursts) is found in tricultures initiated with day 4 iNGN2 progeny, day 46 iSOX9 astrocytes, and day 24 iSOX10 OPCs compared to monocultures containing only iNGN2 progeny.

TABLE 1
Medium composition

| Medium name | Basal medium | Supplements |
|---|---|---|
| mTESR medium | mTESR1 Basal | mTESR1 supplement (StemCell) |
| | Medium (StemCell) | |
| Essential 8 | Essential 8 Flex Basal | Essential 8 Flex supplement (Gibco), 1× |
| medium | medium (Gibco) | Penicillin/Streptomycin (Gibco) |
| Base medium | 1:1 mixture of | 0.5× N2 (Gibco), 0.5× B27 (Gibco), 50 U/ml |
| (neural | Neurobasal medium | Pen-strep (Life Technology), 0.5× GlutaMAX- |
| maintenance | (Gibco) and DMEM- | I (Gibco), 50 uM 2-Mercaptoethanol (Gibco), |
| medium, NMM) | F12 GlutaMAX | 0.5× MEM-NEAA (Gibco), 0.5× Sodium |
| | (Gibco) | Pyruvate (Gibco), 0.025% Human Insulin |
| | | solution (Sigma) |
| Neural induction | NMM | 1 uM LDN193189 (Milteny) and 10 uM |
| medium | | SB431542 (Tocris) |

Neurons

| | | |
|---|---|---|
| NBM medium | 1:1 mixture of | 0.5× N2 (Gibco), 0.5× B27 without Vit. A |
| | Neurobasal medium | (Gibco), GlutaMAX-I (Gibco), Penstrep (Life |
| | and DMEM-F12 | Technology) |
| | GlutaMAX | |

Astrocytes

| | | |
|---|---|---|
| Maturation | 1:1 mixture of | 1× N2 (Gibco), 1× Sodium Pyruvate (Gibco), |
| medium | Neurobasal medium | 1× GlutaMAX (Gibco), 500 uM N-acetyl-L- |
| | and DMEM-F12 | cysteine, 0.1 mM CAMP (Sigma), 10 ng/ml |
| | GlutaMAX | CNTF (Peprotech), 10ng/ml BMP4 |
| | | (Peprotech), 5 ng/ml HB-EGF (Peprotech) |

Oligodendrocytes

| | | |
|---|---|---|
| Oligodendrocyte | DMEM-F12 GlutaMAX | 0.5× N2 (Gibco), 0.5× B27 without Vit. A |
| basal medium | | (Gibco), 50 U/ml Pen-strep (Life Technology), |
| | | 50 uM 2-Mercaptoethanol (Gibco), 0.5× MEM- |
| | | NEAA (Gibco) |
| Oligodendrocyte | DMEM-F12 GlutaMAX | Oligodendrocyte basal medium, 0.1 μMRA |
| induction medium | | (Sigma), 10 uM SB431542 (Milteny), 1 uM |
| | | LDN-193189 (Tocris) |
| Day 8 medium | DMEM-F12 GlutaMAX | Oligodendrocyte basal medium, 0.1 uM RA, |
| | | 1 uM SAG (Milllipore) |
| Day 14 medium | DMEM-F12 GlutaMAX | Oligodendrocyte basal medium, 10 ng/ml |
| | | PDGFaa (Peprotech), 10 ng/ml IGF1, 5 ng/ml |
| | | HGF (Peprotech), 10 ng/ml NT3 (Peprotech), |
| | | 60 ng/ml T3 (Sigma), 100 ng/ml Biotin |
| | | (Sigma), 1 uM CAMP (Sigma), 2 ug/ml |
| | | doxycycline (Sigma) |

TABLE 2
Antibodies

| | | Primary Antibody | Secondary Antibody |
|---|---|---|---|
| Neuron | Tuj1 | Rabbit anti-Tuj1 | Goat anti-rabbit Alexa |
| markers | | (Abcam; 1/1000) | Fluorophore 647 |
| | | | (Thermo Fisher Scientific) |
| | | Chicken anti-Tuj1 | Goat anti-chicken Alexa |
| | | (Merck; 1/1000) | Fluorophore 555 |
| | | | (Thermo Fisher Scientific) |
| | | Mouse anti-Tuj1 | Goat anti-mouse Alexa |
| | | (Merck; 1/1000) | Fluorophore 488 |
| | | | (Thermo Fisher Scientific) |
| | MAP2 | Rabbit anti-MAP2 | Goat anti-rabbit Alexa |
| | | (Synsys; 1/1500) | Fluorophore 647 |
| | | | (Thermo Fisher Scientific) |
| | Tau | Mouse anti-Tau | Goat anti-mouse Alexa |
| | | (Abcam; 1/500) | Fluorophore 488 |
| | | | (Thermo Fisher Scientific) |
| Astrocyte | S100β | Rabbit anti-S100β | Goat anti-rabbit Alexa |
| markers | | (Abcam; 1/200) | Fluorophore 647 |
| | | | (Thermo Fisher Scientific) |
| | GFAP | Rat anti-GFAP | Goat anti-rat Alexa |
| | | (Thermo Fisher | Fluorophore 555 |
| | | Scientific; 1/1000) | (Thermo Fisher Scientific) |
| | EAAT1 | Rabbit anti-EAAT1 | Goat anti-rabbit Alexa |
| | | (Abcam; 1/200) | Fluorophore 647 |
| | | | (Thermo Fisher Scientific) |
| | ALDH1L1 | Mouse anti- | Goat anti-mouse Alexa |
| | | ALDH1L1 | Fluorophore 488 |
| | | (Abcam; 1/500) | (Thermo Fisher Scientific) |
| Oligoden- | O4 | Mouse anti-O4 | Goat anti-mouse Alexa |
| drocyte | | (R&D systems; | Fluorophore 488 |
| markers | | 1/1000) | (Thermo Fisher Scientific) |
| | MBP | Chicken anti-MBP | Goat anti-chicken Alexa |
| | | (Abcam; 1/75) | Fluorophore 555 |
| | | | (Thermo Fisher Scientific) |
| Pluri- | OCT4 | Rabbit anti-OCT4 | Goat anti-rabbit Alexa |
| potency | | (Santa Cruz, | Fluorophore 647 |
| marker | | 1/100) | (Thermo Fisher Scientific) |
| | SOX2 | Rabbit anti-SOX2 | Goat anti-rabbit Alexa |
| | | (Millipore, 1/100) | Fluorophore 647 |
| | | | (Thermo Fisher Scientific) |
| Neural | Nestin | Rabbit anti-Nestin | Goat anti-rabbit Alexa |
| progenitor | | (Biolegend, 1/200) | Fluorophore 647 |
| marker | | | (Thermo Fisher Scientific) |

TABLE 3
Composition of master mediums
Coculture medium

| | | |
|---|---|---|
| Master | 1:1 mixture | 0.5× N2 (Gibco), 0.5× B27 (Gibco), |
| medium 1 | of | 50 U/ml Pen-strep (Life Technology), |
| | Neurobasal | 0.5× GlutaMAX-I (Gibco), 50 μM2- |
| | medium and | Mercaptoethanol (Gibco), 0.5× MEM-NEAA |
| | DMEM-F12 | (Gibco), 0.5× Sodium Pyruvate (Gibco), |
| | GlutaMAX | 0.025% Human Insulin solution (Sigma) |
| Master | 1:1 mixture | 0.5× N2 (Gibco), 0.5× B27 (Gibco), |
| medium 2 | of | 50 U/ml Pen-strep (Life Technology), |
| | Neurobasal | 0.5× GlutaMAX-I (Gibco), 50 μM2- |
| | medium and | Mercaptoethanol (Gibco), 0.5× |
| | DMEM-F12 | MEM-NEAA (Gibco), 0.5× Sodium |
| | GlutaMAX | Pyruvate (Gibco), 1 ug/ml Human Insulin |
| | | solution (Sigma), 1 uMcAMP (SIGMA), |
| | | 100 ng/ml Biotin (SIGMA), 20 ug/ml |
| | | Ascorbate (Sigma), 60 ng/ml T3 (Sigma) |

TABLE 4
Cell ratios and density used to set up triculture system with cortical
NPCs, iSOX9-astrocytes and iSOX10-OPCs

| | | |
|---|---|---|
| Cell ratios | | |
| (NPC:OPC: | | Antibody |
| Astrocytes) | Cell densities/well | combinations |

Cortical neuron based triculture system

| | | |
|---|---|---|
| 1:1:1 | 30,000 cells, 10,000 cells, | Tuj1 + S100β + MBP |
| | 5,000 cells, 2,500 cells | Tuj1 + S100β + O4 |
| | per type | Tuj1 + GFAP + MBP |
| 1:0.5:0.25 | 10,000 neurons, 5,000 | MAP2 + ALDH1L1 + MBP |
| | oligodendrocytes, 2500 | Tau + EAAT1 + O4 |
| | astrocytes | Different layer neuron |
| 1:1:0.5 | 10,000 neurons, 10,000 | markers |
| (for deep | oligodendrocytes, 5,000 | CTIP2 + S100β + MBP |
| layer | astrocytes | SATB2 + S100β + MBP |
| neuron | | |
| markers) | | |
| 1:0.5:0.5 | 10,000 neurons, 5,000 | |
| (for deep | oligodendrocytes, 5,000 | |
| layer | astrocytes | |
| neuron | | |
| markers) | | |

iNGN2 based triculture system

| | | |
|---|---|---|
| 1:1:0.5 | 10,000 neurons, 10,000 | Tuj1 + S100β + MBP |
| (co- and | oligodendrocytes, 5,000 | Tuj1 + S100β + O4 |
| sequential | astrocytes | |
| seeding) | | |
| 1:0.5:0.25 | 10,000 neurons, 5,000 | |
| | oligodendrocytes 2,500 | |
| | astrocytes | |

MEA system

| | | |
|---|---|---|
| 1:0.5:0.5 | 30,000 neurons, 15,000 | Neuron monoculture |
| | oligodendrocytes, 15,000 | 30,000 cells, 50,000 |
| | astrocytes | cells and 75,000 cells |
| | 50,000 neurons, 25,000 | |
| | oligodendrocytes, 25,000 | |
| | astrocytes | |
| | 75,000 neurons, 37,500 | |
| | oligodendrocytes, 37,500 | |
| | astrocytes | |

1. An in vitro cell culture comprising:
neuronal cells derived from iPSC, optionally with an NGN2 transgene,
astrocytes derived from iPSC with a SOX9 transgene, and
oligodendrocytes derived from iPSC with a SOX10 transgene.

2. The in vitro cell culture according to claim 1, comprising neuronal cells derived from iPSC with an NGN2 transgene,

3. The in vitro cell culture according to claim 1 or 2, wherein the neuronal cells express one or more of NESTIN, TUJ1, TBR2, CTIP2, NGN2 and MAP2.

4. The in vitro cell culture according to any one of claims 1 to 3, wherein the neuronal cells express one or more of NESTIN, TUJ1, TBR2, CTIP2, NGN2 and MAP2.

5. The in vitro cell culture according to any of claims 1 to 4, wherein the neuronal cells express one or more of OCT4, NESTIN, TUJ1, TBR2, CTIP2, GABA, glutamate, AcH, NGN2 and MAP2.

6. The in vitro cell culture according to claim 1, wherein the astrocytes express one or more of SOX9, S100β, ALDH1L1, EAAT1, ALDH1L1 and GFAP.

7. The in vitro cell culture according to claim 1 or 6, wherein the astrocytes express one or more of OCT4, NESTIN, SOX9, S100β, ALDH1L1, EAAT1, ALDH1L1 and GFAP.

8. The in vitro cell culture according to claim 1, wherein the oligodendrocytes express one or more of MBP, OLIG1, OLIG2, NKX2.2, SOX10, O4 and MBP.

9. The in vitro cell culture according to claim 1 or 9, wherein the oligodendrocytes express one or more of MBP, OLIG1, OLIG2, NKX2.2, SOX10, O4, PLP4 and MBP.

10. The in vitro cell culture according to claim 1, 8 or 9, wherein the oligodendrocytes express one or more of OCT4, NESTIN, MBP, OLIG1, OLIG2, NKX2.2, SOX10, O4 and MBP.

11. The in vitro cell culture according to any one of claims 1 to 10, expressing CTIP2 and SATB2.

12. The in vitro cell culture according to any one of claims 1 to 11, showing neuronal activity.

13. The in vitro cell culture according to any one of claims 1 to 12, which is a three-dimensional cell culture.

14. A multi electrode array (MEA) system comprising an in vitro culture according to any one of claims 1 to 13.

15. Use of the in vitro cell culture of any one of claims 1 to 13, or the MEA system according to claim 14, in the testing of pharmaceutical compounds.

16. A method of preparing an in vitro culture of iPSC derived neuronal cells, iPSC derived astrocytes and iPSC derived oligodendrocytes, the method comprising the steps of:
a) inducing iPSC, optionally transfected with an inducible NGN2 transgene, into neuronal cells,
b) inducing iPSC, transfected with an inducible SOX9 transgene, into astrocytes,
c) inducing iPSC, transfected with an inducible SOX10 transgene, into oligodendrocytes,
d) cultivating the neuronal cells for between 31 and 45 days,
e) cultivating the astrocytes for between 20 and 46 days,
f) cultivating the oligodendrocytes for between 17 and 31 days,
g) combining the neuronal cells, astrocytes and oligodendrocytes obtained in respectively step d, e, and f, and further cultivating the combined cells for at least 2 weeks and up to 5 weeks.

17. The method according to claim 16, wherein the combined cells are grown as a three-dimensional cell culture.

18. The method according to claim 16 or 17, further comprising the step of detecting the expression of TUJ1, S100beta, MBP and 04, in the combined culture of step g) after one week.

19. The method according to any one of claims 16 to 18, wherein in step g) all of neuronal cells, astrocytes and oligodendrocytes are combined simultaneously.

20. The method according to any one of claims 16 to 18, wherein in step g) neuronal cells, and oligodendrocytes are combined simultaneously and astrocytes are added after between 5 to 8 days.

21. The method according to any one of claims 16 to 20, wherein in step g) wherein between 5000 and 15000 neuronal cells, between 5000 and 15000 oligodendrocyte cells and between 1500 and 7500 astrocytes are combined.

22. The method according to any one of claims 16 to 21, wherein the cultivated combined cells of step g) are assayed for the expression of CTIP2 and SATB2.

23. The method according to any one of claims 16 to 22, wherein the cultivated combined cells of step g) are tested for neuronal activity by measuring neuronal network electrical activity with multielectrode array.
